(19) United States
(12) Patent Application Publication
(10) Pub. No.: US 2008/0199897 A1
(43) Pub. Date: Aug. 21, 2008

(54) CLASS II HISTONE DEACETYLASE WHOLE CELL ENZYME ASSAY

(75) Inventors: Jubrail Rahil, Dollard Des Ormeauz (CA); Aihua Lu, Montreal (CA)

Correspondence Address: KEOWN & ZUCCHERO, LLP, 500 WEST CUMMINGS PARK, SUITE 1200, WOBURN, MA, 01801, US

(73) Assignee: MethylGene Inc.

(21) Appl. No.: 12/025,918

(22) Filed: Feb. 5, 2008

Related U.S. Application Data
(60) Provisional application No. 60/888,205, filed on Feb. 5, 2007.

(51) Int. Cl. C12Q 1/37; C07D 311/00; C12N 9/48

(52) U.S. Cl. 435/23; 549/399; 435/212

(57) ABSTRACT

The invention relates to enzymatic assays and substrates for Class II histone deacetylases. More particularly, the invention relates to such assays and substrates utilizing whole cells, extracts of such whole cells, extracts of sub-cellular compartments of such whole cells, or bodily fluids.

### CROSS-REFERENCE TO RELATED APPLICATIONS

This application claims the benefit of priority of U.S. Application No. 60/888,205, filed Feb. 5, 2007, the contents of which are incorporated herein by reference.

### BACKGROUND OF THE INVENTION

1. Field of the Invention

The invention relates to enzymatic assays and substrates for protein deacetylases. More particularly, the invention relates to such assays and substrates utilizing primary intact whole cells.

2. Summary of the Related Art

Histone deacetylases play an important role in gene regulation in mammalian cells. Gray and Ekstrom, Expr. Cell. Res. 262: 75-83 (2001); Zhou et al., Proc. Natl. Acad. Sci. USA 98: 10572-10577 (2001); Kao et al. J. Biol. Chem. 277: 187-193 (2002) and Gao et al. J. Biol. Chem. 277: 25748-25755 (2002) teach that there are 11 members of the histone deacetylase (HDAC) family. Another family of deacetylases involved in gene expression is the Sir2 family. Gray and Ekstrom, supra, teach that there are seven members of the Sir2 family in humans.

Class I histone deacetylases include HDAC 1, HDAC2, HDAC3 and HDAC8. The Class I enzymes are expressed in a wide variety of tissues and are reported to be localized in the nucleus. Class II histone deacetylases include HDAC4, HDAC5, HDAC6, HDAC7, HDAC9 and HDAC10. The Class II enzymes have been described as limited in tissue distribution and they can shuttle between the nucleus and the cytoplasm. The Class II enzymes are further divided into Class IIa (HDAC4, HDAC5, HDAC7 and HDAC9) and Class IIb (HDAC6 and HDAC10). Recent classifications place HDAC11 in a class of its own.

The role of HDACs in transcription and its link to diseases, such as cancer has recently been explored. Minnucci et al., Proc. Natl. Acad. Sci. USA 94: 11295-11300 (1997); Hassig et al., Chem. Biol. 4: 783-789 (1998); Grignani et al., Nature 391: 815-818 (1998) and Siddique et al., Oncogene 16: 2283-2285 (1998) suggest that inhibitors of HDACs may be useful for transcription therapy in various human diseases.

As efforts at developing HDAC inhibitors for therapeutic treatment progresses, there is a need for assays to determine the activity of such inhibitors. Lechner et al., Biochim. Biophys. Acta 1296: 181-188 (1996) teaches the use of tritylated, acetylated histones as a substrate. Taunton et al., Science 272: 408-411 (1996) teaches the use of tritylated, acetylated synthetic peptides derived from histones as substrate. These assays proved difficult to standardize.

More recently, non-radioisotopic assays have been developed. Heltweg and Jung, Journal of Biomolecular Screening 8: 89-95 (2003) describes an assay using the fluorescent compound MAL (Boc-LysAc-AMC) and a partially purified rat liver HDAC in the presence or absence of the HDAC inhibitor trichostatin A. Heltweg B et al. Analytical Biochemistry (2003) also disclosed the use of the same small molecule substrate and its derivative for several recombinant HDAC isotypes in vitro. Wegener et al., Chemistry & Biology 10: 61-68 (2003) disclosed the use of fluorogenic HDAC substrates with an acetylated lysine, which upon deacetylation becomes a substrate for trypsin and then releases the fluorophore. Similarly, Biomol (Plymouth Meeting, Philadelphia) disclosed several fluorescent activity kits which could monitor HDAC activities in vitro (“HDAC Fluorescent Activity/Drug Discovery Kit”) or could specifically monitor SirT1, SirT2 or SirT3 activity in vitro. In vitro by using recombinant enzymes, inhibitory activity of suramin as well as activator activity of resverastrol could be monitored against Sirtuins and inhibitory activity of TSA could be monitored against HDACs in extracts or recombinant HDAC isotypes. Unfortunately, these and similar assays all require forming cellular extracts, which is time consuming and may result in artifacts from the extraction procedure.

The “HDAC Fluorescent Activity/Drug Discovery Kit” (Biomol) discloses an assay using cultured HeLa and Jurkat whole cells using an undisclosed acetylated HDAC (class I/II) pan-substrate that generates a fluorescent reporter molecule and measuring fluorescent HDAC cleavage product in the wells in which the cells were cultured. However, methods are lacking to measure 1) potency and isotype-specificity of a given class I/II HDAC inhibitor in whole cell context; 2) potency and isotype-specific of a Sirtuin inhibitors in whole cell context; and 3) HDAC activity from primary cells taken from a mammal or a mammal treated with HDAC class I/II inhibitors or sirtuin inhibitors. Especially in the latter scenario, primary whole cells taken from a mammal may not be susceptible to culturing and such cultured cells may not reflect the actual activity of HDAC in the cells in the body of the mammal.

Co-pending U.S. Ser. No. 11/231,528, filed Sep. 21, 2005, discloses a whole cell assay for protein deacetylase activity, including histone deacetylase activity. However, this disclosure does not discriminate between Class I HDACs and Class II HDACs.

There is therefore a need for assays and substrates which allow assessment of 1) class selectivity of HDAC inhibitors, preferably in a whole cell context and 2) level of Class I and/or Class II histone deacetylase activity in a cell, preferably a whole cell taken directly from the body of the mammal.

### BRIEF SUMMARY OF THE INVENTION

The invention provides assays and substrates which allow assessment of the level of Class II histone deacetylase activity in a cell, for example in primary intact whole cells taken directly from the body of a mammal, or from bodily fluids, extracts from such cells, or extracts of sub-cellular compartments from such cells.

In a first aspect, the invention provides a method for assessing Class II histone deacetylase activity or activity of one or more member thereof in whole cells ex vivo, or in extracts of such cells or in extracts of subcellular compartments from such cells. In the method according to this aspect of the invention whole cells, preferably from a mammal, or bodily fluids, extracts from such cells, or extracts of sub-cellular compartments from such cells, are provided and contacted with a Class II histone deacetylase-specific substrate, wherein deacetylation of the substrate by Class II histone deacetylases or one or more member thereof generates a detectable reporter molecule. The quantity of the detectable reporter molecule is then measured either in the whole cells, bodily fluids, or in extracts from such cells or extracts from subcellular compartments of such cells. In preferred embodiments, the quantity of the detectable reporter molecule is measured against a control standard for the Class II histone deacetylase family or the one or more member thereof. In preferred embodiments, the substrate is cell-permeable.

In a second aspect, the invention provides a method for assessing Class II histone deacetylase activity, or activity of one or more member thereof in a mammal in vivo by measuring the quantity of a detectable reporter molecule in bodily fluids. In the method according to this aspect of the invention, the mammal is administered a cell-permeable Class II histone deacetylase-specific substrate, wherein deacetylation of the substrate by Class II histone deacetylases, or one or more member thereof generates a detectable reporter molecule. Bodily fluids from the mammal are obtained and the quantity of the detectable reporter molecule in the bodily fluids is determined. In a preferred embodiment, the quantity of the detectable reporter molecule is measured against a control standard for the Class II histone deacetylase family or the one or more member thereof.

In a third aspect, the invention provides a method for assessing isotype-specific activity of one or more member of the Class II histone deacetylase family in whole cells ex vivo, or in extracts from such cells or extracts from subcellular compartments of such cells, In the method according to this aspect of the invention whole cells from a mammal, or extracts from such cells or extracts from subcellular compartments of such cells, are provided and contacted with a Class II HDAC-specific substrate or a isotype-specific substrate for the one or more member of the Class II HDAC family, wherein deacetylation of the substrate by the one or more Class II HDAC generates a detectable reporter molecule. A first aliquot of the cells, or said extracts, is further contacted with an isotype-specific inhibitor of the one or more Class II HDAC and a second aliquot of the cells is not. The quantity of the detectable reporter molecule is then measured for the first and second aliquots and the quantity of HDAC activity for each aliquot is compared. In preferred embodiments, the quantity of the detectable reporter molecule is measured against a control standard for the Class II HDAC family or the one or more member thereof. In preferred embodiments, the substrate is cell-permeable.

In a fourth aspect, the invention provides a method for assessing isotype-specific activity of one or more member of the Class II histone deacetylase family in a mammal in vivo by measuring the quantity of a detectable reporter molecule in bodily fluids, In the method according to this aspect of the invention the mammal is administered a cell-permeable Class II HDAC-specific substrate or a cell permeable isotype-specific substrate for the one or more member of the Class II HDAC family, wherein deacetylation of the substrate by the one or more member of the Class II HDAC family generates a detectable reporter molecule. A first sample of bodily fluid is obtained and then the mammal is further administered an isotype-specific inhibitor of the one or more Class II HDAC and a second sample of bodily fluid is obtained. The quantity of the detectable reporter molecule is then measured for the first and second samples and the quantity of HDAC activity for each sample is compared. In preferred embodiments, the quantity of the detectable reporter molecule is measured against a control standard for the Class II HDAC family or the one or more member thereof.

In a fifth aspect, the invention provides a method for assessing the activity of one or more specific isotype of the Class II HDAC family in cells ex vivo, in extracts of such cells, or in extracts of sub-cellular compartments of such cells. In the method according to this aspect of the invention whole cells, preferably from a mammal, or extracts from such cells or extracts from subcellular compartments of such cells are provided and contacted with a isotype-specific substrate for the one or more particular member of the Class II HDAC family, wherein deacetylation of the substrate by the HDAC generates a detectable reporter molecule and measuring the quantity of the detectable reporter molecule. In preferred embodiments, the quantity of the detectable reporter molecule is measured against a control standard for the Class II HDAC family or the one or more member thereof. In preferred embodiments, the substrate is cell-permeable.

In a sixth aspect, the invention provides a method for assessing the activity of a candidate Class II HDAC-specific inhibitor or an inhibitor of one or more member thereof in whole cells ex vivo, in extracts of such cells, or in extracts of sub-cellular compartments of such cells. In the method according to this aspect of the invention whole cells, preferably from a mammal, or extracts from such cells or extracts from subcellular compartments of such cells, are provided and contacted with a Class II HDAC-specific substrate, wherein deacetylation of the substrate by the Class II HDAC family or one or more members thereof generates a detectable reporter molecule. A first aliquot of the cells, or said extracts, is further contacted with the candidate Class II HDAC-specific inhibitor or candidate inhibitor of one or more member thereof and a second aliquot of the cells, or said extracts, is not. The quantity of the detectable reporter molecule is then measured for the first and second aliquots and the quantity of protein deacetylase activity for each aliquot is compared. In preferred embodiments, the quantity of the detectable reporter molecule is measured against a control standard for the protein deacetylase family or the one or more members thereof. In preferred embodiments, the substrate is cell-permeable.

In a seventh aspect, the invention provides a method for assessing isotype-specific activity of a candidate inhibitor of a member of the Class II HDAC family in whole cells ex vivo, in extracts of such cells, or in extracts of sub-cellular compartments of such cells. In the method according to this aspect of the invention whole cells, preferably from a mammal, or extracts from such cells or extracts from subcellular compartments of such cells, are provided and contacted with a Class II HDAC-specific substrate or an isotype-specific substrate for one or more member of the Class II HDAC family, wherein deacetylation of the substrate by the protein deacetylase generates a detectable reporter molecule. A first aliquot of the cells, or said extracts, is further contacted with the candidate isotype-specific inhibitor of the member of the Class II HDAC family and a second aliquot of the cells, or said extracts, is not. The quantity of the detectable reporter molecule is then measured for the first and second aliquots and the quantity of the detectable reporter molecule for each aliquot is compared. In preferred embodiments, the quantity of the detectable reporter molecule is measured against a control standard for the protein deacetylase family or the one or more member thereof. In preferred embodiments, the substrate is cell-permeable.

In an eighth aspect, the invention provides a method for assessing the efficacy of a Class II HDAC-specific inhibitor or an inhibitor of one or more member thereof in vivo. In the method according to this aspect of the invention, whole cells are provided, from a mammal. The cells are contacted with a cell permeable Class II HDAC-specific substrate or a cell permeable isotype specific substrate, wherein deacetylation of the substrate by the Class II HDAC family or one or more members thereof generates a detectable reporter molecule. The quantity of the reporter molecule is then determined. In preferred embodiments, the quantity is standardized against a known activity of the Class II HDAC or one or more member thereof. Next, the mammal is administered the Class II HDAC-specific inhibitor or the inhibitor of one or more member thereof. After an appropriate period of time, whole cells are again taken from the mammal and contacted with the Class II HDAC-specific substrate. Next the quantity of the reporter molecule is determined. In preferred embodiments, the quantity is standardized against a known activity of the Class II HDAC or the one or more members thereof. Then the quantity of the reporter molecule after administration of the Class II HDAC-specific inhibitor, or inhibitor of one or more member thereof, is compared with the quantity of the reporter molecule before administration of the Class II HDAC-specific inhibitor, or inhibitor of one or more member thereof. Significant decrease in the quantity of the reporter molecule after administration of the Class II HDAC-specific inhibitor, or inhibitor of one or more member thereof, is taken as a measure of efficacy.

In a ninth aspect, the invention provides a method for assessing the efficacy and specificity of an isotype-specific inhibitor of one or more member of the Class II HDAC family in vivo. In the method according to this aspect of the invention, whole cells are provided from a mammal. The cells are contacted with a cell permeable isotype-specific substrate for the one or more member of the Class II HDAC family, wherein deacetylation of the substrate by the HDAC generates a detectable reporter molecule. The quantity of the reporter molecule is then determined. In preferred embodiments, the quantity is standardized against a known activity of the one or more member of the Class II HDAC family. Next, the mammal is administered the isotype-specific inhibitor. After an appropriate period of time, whole cells are again taken from the mammal and contacted with the isotype-specific substrate. Next the quantity of the reporter molecule is determined. In preferred embodiments, the quantity is standardized against a known activity of the one or more member of the Class II HDAC family. Then the quantity of the reporter molecule after administration of the isotype-specific inhibitor is compared with the quantity of the reporter molecule before administration of the isotype-specific inhibitor. Significant decrease in the quantity of the reporter molecule after administration of the isotype-specific inhibitor is taken as a measure of efficacy.

In a tenth aspect, the invention provides a method for assessing the efficacy of a Class II HDAC-specific inhibitor or an inhibitor of one or more member thereof in vivo by measuring the quantity of a detectable reporter molecule in bodily fluids from a mammal. In the method according to this aspect of the invention, the mammal is administered a cell-permeable Class II HDAC-specific substrate or an isotype-specific substrate, wherein deacetylation of the Class II HDAC-specific substrate or isotype-specific substrate generates a detectable reporter molecule. Bodily fluids from the mammal are obtained and the quantity of the detectable reporter molecule in the bodily fluids is determined. The mammal is then administered the Class II HDAC-specific inhibitor, or inhibitor of one or more member thereof, and after an appropriate time period the mammal is administered the Class II HDAC-specific substrate. Bodily fluids from the mammal are obtained and the quantity of the detectable reporter molecule in the bodily fluids is determined. The quantity of detectable reporter molecule in bodily fluids obtained prior to administration of the Class II HDAC-specific inhibitor, or inhibitor of one or more member thereof, is then compared with the quantity of the detectable reporter molecule in bodily fluids after administration of the Class II HDAC-specific inhibitor, or inhibitor of one or more member thereof. Significant decrease in the quantity of the reporter molecule after administration of the inhibitor is taken as a measure of efficacy.

In an eleventh aspect, the invention provides a method for assessing the efficacy of an isotype-specific inhibitor of one or more member of the Class II HDAC family in mammals in vivo by measuring the quantity of a detectable reporter molecule in bodily fluids. In the method according to this aspect of the invention, the mammal is administered a cell-permeable isotype-specific substrate for the one or more member of the Class II HDAC family, wherein deacetylation of the isotype-specific substrate generates the detectable reporter molecule. Bodily fluids from the mammal are obtained and the quantity of the detectable reporter molecule in the bodily fluids is determined. The mammal is then administered an isotype-specific inhibitor of one or more member of the Class II HDAC family and after an appropriate time period the mammal is administered the isotype-specific substrate. Bodily fluids from the mammal are obtained and the quantity of the detectable reporter molecule in the bodily fluids is determined. The quantity of detectable reporter molecule in bodily fluids obtained prior to administration of the isotype-specific inhibitor is then compared with the quantity of the detectable reporter molecule in bodily fluids after administration of the isotype-specific inhibitor. Significant decrease in the quantity of the reporter molecule after administration of the isotype-specific inhibitor is taken as a measure of efficacy.

In a twelfth aspect, the invention provides a method for assessing the efficacy of a Class II HDAC-specific activator or an activator of one or more member thereof in vivo. In the method according to this aspect of the invention, whole cells are provided from a mammal. The cells are contacted with a cell permeable Class II HDAC-specific substrate or a cell permeable isotype specific substrate, wherein deacetylation of the substrate by the Class II HDAC family or one or more members thereof generates a detectable reporter molecule. The quantity of the reporter molecule is then determined. In preferred embodiments, the quantity is standardized against a known activity of the Class II HDAC family or the one or more members thereof. Next, the mammal is administered the Class II HDAC-specific activator, or the activator of one or more member thereof. After an appropriate period of time, whole cells are again taken from the mammal and contacted with the Class II HDAC-specific substrate, or isotype specific substrate. Next the quantity of the reporter molecule in the whole cells is determined. In preferred embodiments, the quantity is standardized against a known activity of the Class II HDAC family or the one or more members thereof. Then the quantity of the reporter molecule after administration of the Class II HDAC-specific activator, or activator of one or more member thereof, is compared with the quantity of the reporter molecule before administration of the Class II HDAC-specific activator, or activator of one or more member thereof. Significant increase in the quantity of the reporter molecule after administration of the Class II HDAC-specific activator, or activator of one or more member thereof, is taken as a measure of efficacy.

In a thirteenth aspect, the invention provides a method for assessing the efficacy and specificity of an isotype-specific activator of one or more member of the Class II HDAC family in vivo. In the method according to this aspect of the invention, whole cells are provided from a mammal. The cells are contacted with a cell permeable isotype-specific substrate for the one or more member of the Class II HDAC family, wherein deacetylation of the substrate by the HDAC generates a detectable reporter molecule. The quantity of the reporter molecule is then determined. In preferred embodiments, the quantity is standardized against a known activity of the one or more member of the Class II HDAC family. Next, the mammal is administered the isotype-specific activator. After an appropriate period of time, whole cells are again taken from the mammal and contacted with the isotype-specific substrate. Next the quantity of the reporter molecule is determined. In preferred embodiments, the quantity is standardized against a known activity of the one or more member of the Class II HDAC family. Then the quantity of the reporter molecule after administration of the isotype-specific activator is compared with the quantity of the reporter molecule before administration of the isotype-specific activator. Significant increase in the quantity of the reporter molecule after administration of the isotype-specific activator is taken as a measure of efficacy.

In a fourteenth aspect, the invention provides a method for assessing the efficacy of a Class II HDAC-specific activator or an activator of one or more members thereof in vivo by measuring the quantity of a detectable reporter molecule in bodily fluids from a mammal. In the method according to this aspect of the invention, the mammal is administered a cell-permeable Class II HDAC-specific substrate or substrate for one or more members thereof, wherein deacetylation of the Class II HDAC-specific substrate or isotype-specific substrate generates the detectable reporter molecule. Bodily fluids from the mammal are obtained and the quantity of the detectable reporter molecule in the bodily fluids is determined. In preferred embodiments, the quantity is standardized against a known activity of the one or more member of the Class II HDAC family. The mammal is then administered the Class II HDAC-specific activator, or activator of one or more members thereof, and after an appropriate time period the mammal is administered the Class II HDAC-specific substrate or isotype specific substrate. Bodily fluids from the mammal are obtained and the quantity of the detectable reporter molecule in the bodily fluids is determined. In preferred embodiments, the quantity is standardized against a known activity of the one or more member of the Class II HDAC family. The quantity of detectable reporter molecule in bodily fluids obtained prior to administration of the Class II HDAC-specific activator, or activator of one or more member thereof, is then compared with the quantity of the detectable reporter molecule in bodily fluids after administration of the Class II HDAC-specific activator, or activator of one or more member thereof. Significant increase in the quantity of the reporter molecule after administration of the Class II HDAC-specific activator, or activator of one or more member thereof, is taken as a measure of efficacy.

In a fifteenth aspect, the invention provides a method for assessing the efficacy of an isotype-specific activator of one or more member of the Class II HDAC family in a mammal in vivo by measuring the quantity of a detectable reporter molecule in bodily fluids from the mammal. In the method according to this aspect of the invention, the mammal is administered a cell-permeable isotype-specific substrate for a Class II HDAC, or one or more member thereof, wherein deacetylation of the isotype-specific substrate generates the detectable reporter molecule. Bodily fluids from the mammal are obtained and the quantity of the detectable reporter molecule in the bodily fluids is determined. In preferred embodiments, the quantity is standardized against a known activity of the one or more member of the Class II HDAC family. The mammal is then administered an isotype-specific activator of one or more member of the Class II HDAC family and after an appropriate time period the mammal is administered the isotype-specific substrate. Bodily fluids from the mammal are obtained and the quantity of the detectable reporter molecule in the bodily fluids is determined. In preferred embodiments, the quantity is standardized against a known activity of the one or more member of the Class II HDAC family. The quantity of detectable reporter molecule in bodily fluids obtained prior to administration of the isotype-specific activator is then compared with the quantity of the detectable reporter molecule in bodily fluids after administration of the isotype-specific activator. Significant increase in the quantity of the reporter molecule after administration of the isotype-specific activator is taken as a measure of efficacy.

In a sixteenth aspect, the invention provides a method for assessing the activity of a candidate Class II HDAC-specific activator or an activator of one or more members thereof in whole cells ex vivo. In the method according to this aspect of the invention whole cells from a mammal are provided and contacted with a cell-permeable a Class II HDAC-specific substrate or an isotype-specific substrate, wherein deacetylation of the substrate by the Class II HDAC family or one or more members thereof generates a detectable reporter molecule. A first aliquot of the cells is further contacted with a candidate Class II HDAC-specific activator, or an activator of one or more member thereof, and a second aliquot of the cells is not. The quantity of the detectable reporter molecule is then measured for the first and second aliquots and the quantity of Class II HDAC-specific activity, or activity of one or more member thereof, for each aliquot is compared. In preferred embodiments, the quantity of the detectable reporter molecule is measured against a control standard for the protein deacetylase family or the one or more members thereof.

In a seventeenth aspect, the invention provides a method for assessing the activity of a candidate isotype-specific activator of a member of the Class II HDAC family ex vivo. In the method according to this aspect of the invention whole cells from a mammal are provided and contacted with a cell-permeable isotype-specific substrate, wherein deacetylation of the substrate by the member of the Class II HDAC family generates a detectable reporter molecule. A first aliquot of the cells is further contacted with a candidate isotype-specific activator of the member of the Class II HDAC family and a second aliquot of the cells is not. The quantity of the detectable reporter molecule is then measured for the first and second aliquots and the quantity of isotype-specific HDAC activity for each aliquot is compared. In preferred embodiments, the quantity of the detectable reporter molecule is measured against a control standard for the member of the Class II HDAC family.

In an eighteenth aspect, the invention provides compounds of formula (I), with the proviso that the compound is not Boc-Lys(Ac)-AMC or Boc-Lys(Tfa)-AMC:

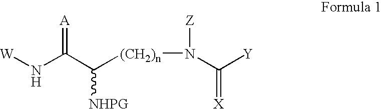

wherein
X is selected from the group consisting of O, S, NH and N(alkyl);
Y is selected from the group consisting of OH, alkoxy, alkyl, alkenyl and alkynyl, each of which alkoxy, alkyl, alkenyl and alkynyl, is optionally substituted with 1 to 7 substituents independently selected from the group consisting of halo, cyano, alkoxy, alkylamino and alkylthio;
Z is selected from the group consisting of H, alkyl, alkenyl and alkynyl;
n is an integer ranging from 0 to 12;
PG is a protecting group (preferably selected from the group consisting of MeCO, CF₃CO—, Boc and CBZ), an amino acide or a peptide;
A is selected from the group consisting of O, S, NH and N(alkyl); and
W is a carboxycylic, heterocyclic, saturated or unsaturated, aromatic or heteroaromatic mono-, bi-, tri- or tetracyclic ring system (preferably a mono- or bicyclic aryl or a mono- or bicyclic heteroaryl ring system).

In another aspect, the invention provides for the use of a compound according to Formula I as a substrate for Class II histone deacetylases.

In another aspect, the invention provides a complex of a compound according to Formula I bound to a Class II histone deacetylase.

### BRIEF DESCRIPTION OF THE DRAWINGS

FIG. 1a shows intracellular and excellular HDAC activity in cultured 293T cells using the Boc-Lys(Ac)-AMC substrate; FIG. 1b shows HDAC Class II activity in 239TV cells using Boc-Lys(Tfa)-AMC substrate.

FIG. 2 shows a scheme for generation of a detectable reporter molecule for a representative cell permeable substrate.

FIG. 3a shows whole cell HDAC activity as a function of cell numbers in cultured human cancer cells and normal cells using the Boc-Lys(Ac)-AMC substrate; FIG. 3b shows whole cell HDAC activity as a function of cell numbers in cultured human cancer cells and normal cells using the Boc-Lys(Tfa)-AMC substrate.

FIG. 4a shows the effect of Boc-Lys(Ac)-AMC substrate concentration on HDAC whole cell activity in human cancer cell lines; FIG. 4b shows the effect of Boc-Lys(Tfa)-AMC substrate concentration on HDAC whole cell activity in human cancer cell lines.

FIG. 5a shows inhibition of whole cell HDAC activity in human cancer cells by SAHA, Compound 2 and LAQ-824; FIG. 5b shows inhibition of HDAC activity by various compounds using Boc-Lys(Tfa)-AMC substrate.

FIG. 6 shows whole cell HDAC activity as a function of cell numbers in human white blood cells, using Boc-Lys(Ac)-AMC as substrate.

FIG. 7 shows dose-dependent inhibition of whole cell HDAC activity in human white blood cells by HDAC inhibitors (Compound 2 and LAQ-824); as well as their isotypic enzyme inhibitory activities, using Boc-Lys(Ac)-AMC as substrate.

FIG. 8 shows time-dependent inhibition of HDAC enzyme activity in white blood cells from mice treated with Compound 2 using Boc-Lys(Ac)-AMC as substrate.

FIG. 9 shows dose-dependent inhibition of whole cell HDAC activity and histone acetylation in white blood cells from mice treated with Compound 2 using Boc-Lys(Ac)-AMC as substrate.

FIG. 10 shows dose-dependent antitumor activity of Compound 2 in A431 xenograft model in mice using Boc-Lys(Ac)-AMC as substrate.

FIG. 11 shows detection of HDAC activity from serum isolated from mouse whole blood contacted with an HDAC substrate using Boc-Lys(Ac)-AMC as substrate.

FIG. 12 shows three HDAC substrates. Boc-Lys(Ac)-AMC is specific for Class I HDACs, especially HDAC1, HDAC2 and HDAC6. Boc-Lys(Tfa)-AMC and Boc-Lys(thioAc)-AMC are specific for Class II HDACs.

FIG. 13 shows enzymatic reactivity of Boc-Lys(Ac)-AMC and Boc-Lys(Tfa)-AMC for Class I and Class II HDACs.

FIG. 14 shows enzymatic reactivity of Boc-Lys(Ac)-AMC and Boc-Lys(thioAc)-AMC for Class I and Class II HDACs.

FIG. 15 shows whole cell (splenocyte) enzymatic activity of Boc-Lys(Ac)-AMC and Boc-Lys(Tfa)-AMC in the presence or absence of a Class I HDAC-specific inhibitor (Cpd A) or a Class II HDAC-specific inhibitor (Cpd C).

FIG. 16 shows whole cell (three different cell lines) enzymatic activity of Boc-Lys(Ac)-AMC and Boc-Lys(Tfa)-AMC in the presence or absence of a Class I HDAC-specific inhibitor (Cpd A) or a Class II HDAC-specific inhibitor (Cpd C).

FIG. 17 shows whole cell (adipocyte) enzymatic activity of Boc-Lys(Ac)-AMC and Boc-Lys(Tfa)-AMC in the presence or absence of a Class I HDAC-specific inhibitor (Cpd A) or a Class II HDAC-specific inhibitor (Cpd C).

FIG. 18 shows HDAC activity in various cell fractions using Boc-Lys(Ac)-AMC, Boc-Lys(TFA)-AMC, or Boc-Lys(thioAc)-AMC.

FIG. 19 shows anomalous behavior of HDAC 6.

FIG. 20 shows that HDAC 9 behaves like other Class II HDACs.

FIG. 21 shows HDAC additive activity in 293TV cells when measured by Boc-Lys(Ac)-AMC and Boc-Lys(Tfa)-AMC simultaneously, consistent with specificity of each substrate to a different HDAC class.

### DETAILED DESCRIPTION OF THE PREFERRED EMBODIMENTS

The invention relates to enzymatic assays and substrates for protein deacetylases. More particularly, the invention relates to such assays and substrates utilizing whole cells. The invention provides assays and substrates which allow assessment of the level of a protein deacetylase activity in whole cells taken directly from the body of a mammal or in bodily fluids.

In a first aspect, the invention provides a method for assessing Class II histone deacetylase activity or activity of one or more member thereof in whole cells ex vivo, or in extracts of such cells or in extracts of subcellular compartments from such cells. In the method according to this aspect of the invention whole cells, preferably from a mammal, or bodily fluids, extracts from such cells, or extracts of sub-cellular compartments from such cells, are provided and contacted with a Class II histone deacetylase-specific substrate, wherein deacetylation of the substrate by Class II histone deacetylases or one or more member thereof generates a detectable reporter molecule. The quantity of the detectable reporter molecule is then measured either in the whole cells, bodily fluids, or in extracts from such cells or extracts from subcellular compartments of such cells. In preferred embodiments, the quantity of the detectable reporter molecule is measured against a control standard for the Class II histone deacetylase family or the one or more member thereof. In preferred embodiments, the substrate is cell-permeable.

The “Class II histone deacetylase (HDAC) family” is a group of related proteins having the ability to remove acetyl groups from basic side chains of amino acid residues of proteins, such as histones, comprising HDAC4, HDAC5, HDAC7, HDAC9 and HDAC10 but not including HDAC1, HDAC2, HDAC3, HDAC8 or HDAC11, which are regarded as Class I HDACs or Class III HDACs, respectively. Also excluded, for purposes of the invention, is HDAC6, which is generally regarded as a Class II HDAC, but which behaves anomalously in the present methods. The term “mammal” specifically includes humans. “Whole cells” are intact cells, which may be present separately or as part of a tissue or a tumor. “Cell permeable Class II HDAC-specific substrates” are molecules which penetrate cells and which do not provide a detectable reporter molecule in their native form, but which do provide for a detectable molecule after cleavage by one or more members of the Class II HDAC family, without providing for the detectable molecule as a result of a comparable level of cleavage by one or more members of another HDAC family. A “cell permeable isotype-specific inhibitor” is an HDAC inhibitor, or salt thereof, that inhibits one or more member, but less than all members of the Class II HDAC family. A “detectable reporter molecule” is a molecule that provides a measurable signal in an assay. The nature of the molecule is not critical as long as it is measurable. Preferred detectable reporter molecules include, without limitation, colorometric molecules, fluorescent molecules, FRET-detectable molecules, enzymes, radiolabels and chemiluminescent molecules. A “Class II HDAC control standard” is a sample having a known level of Class II HDAC activity.

The whole cells can be contacted with a cell-permeable Class II-HDAC-specific inhibitor or isotype-specific inhibitor alone or in combination with a pharmaceutically acceptable carrier. As used herein, the term “pharmaceutically acceptable” refers to a material that does not interfere with the effectiveness of the assay and is compatible with a biological system such as a cell, tissue, or organism. As used herein, the term “carrier” encompasses any excipient, diluent, filler, salt, buffer, stabilizer, solubilizer, lipid, or other material well known in the art for use in pharmaceutical formulations. It will be understood that the characteristics of the carrier, excipient, diluent etc., will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990.

In a second aspect, the invention provides a method for assessing Class II histone deacetylase activity, or activity of one or more member thereof in a mammal in vivo by measuring the quantity of a detectable reporter molecule in bodily fluids. In the method according to this aspect of the invention, the mammal is administered a cell-permeable Class II histone deacetylase-specific substrate, wherein deacetylation of the substrate by Class II histone deacetylases, or one or more member thereof generates a detectable reporter molecule. Bodily fluids from the mammal are obtained and the quantity of the detectable reporter molecule in the bodily fluids is determined. In a preferred embodiment, the quantity of the detectable reporter molecule is measured against a control standard for the Class II histone deacetylase family or the one or more member thereof.

All definitions are as described above.

In a third aspect, the invention provides a method for assessing isotype-specific activity of one or more member of the Class II histone deacetylase family in whole cells ex vivo, or in extracts from such cells or extracts from subcellular compartments of such cells, In the method according to this aspect of the invention whole cells from a mammal, or extracts from such cells or extracts from subcellular compartments of such cells, are provided and contacted with a Class II HDAC-specific substrate or a isotype-specific substrate for the one or more member of the Class II HDAC family, wherein deacetylation of the substrate by the one or more Class II HDAC generates a detectable reporter molecule. A first aliquot of the cells, or said extracts, is further contacted with an isotype-specific inhibitor of the one or more Class II HDAC and a second aliquot of the cells is not. The quantity of the detectable reporter molecule is then measured for the first and second aliquots and the quantity of HDAC activity for each aliquot is compared. In preferred embodiments, the quantity of the detectable reporter molecule is measured against a control standard for the Class II HDAC family or the one or more member thereof. In preferred embodiments, the substrate is cell-permeable.

One or more isotype may provide a majority of the total protein HDAC activity either naturally, or because the cell has been transfected with the one or more isotype and overexpresses it. The terms “first aliquot” and “second aliquot” are used for convenience and do not imply which aliquot is prepared first temporally. All other definitions are as described above.

In a fourth aspect, the invention provides a method for assessing isotype-specific activity of one or more member of the Class II histone deacetylase family in a mammal in vivo by measuring the quantity of a detectable reporter molecule in bodily fluids, In the method according to this aspect of the invention the mammal is administered a cell-permeable Class II HDAC-specific substrate or a cell permeable isotype-specific substrate for the one or more member of the Class II HDAC family, wherein deacetylation of the substrate by the one or more member of the Class II HDAC family generates a detectable reporter molecule. A first sample of bodily fluid is obtained and then the mammal is further administered an isotype-specific inhibitor of the one or more Class II HDAC and a second sample of bodily fluid is obtained. The quantity of the detectable reporter molecule is then measured for the first and second samples and the quantity of HDAC activity for each sample is compared. In preferred embodiments, the quantity of the detectable reporter molecule is measured against a control standard for the Class II HDAC family or the one or more member thereof.

An “isotype-specific substrate” is a substrate for one or more member, but less than all members of the Class II HDAC family. Certain other isotype-specific substrates include substrates specific for a single member of a Class II HDAC, e.g., HDAC-4. All other definitions are as described above.

In a fifth aspect, the invention provides a method for assessing the activity of one or more specific isotype of the Class II HDAC family in cells ex vivo, in extracts of such cells, or in extracts of sub-cellular compartments of such cells. In the method according to this aspect of the invention whole cells, preferably from a mammal, or extracts from such cells or extracts from subcellular compartments of such cells are provided and contacted with a isotype-specific substrate for the one or more particular member of the Class II HDAC family, wherein deacetylation of the substrate by the HDAC generates a detectable reporter molecule and measuring the quantity of the detectable reporter molecule. In preferred embodiments, the quantity of the detectable reporter molecule is measured against a control standard for the Class II HDAC family or the one or more member thereof. In preferred embodiments, the substrate is cell-permeable. All definitions are as described above.

In a sixth aspect, the invention provides a method for assessing the activity of a candidate Class II HDAC-specific inhibitor or an inhibitor of one or more member thereof in whole cells ex vivo, in extracts of such cells, or in extracts of sub-cellular compartments of such cells. In the method according to this aspect of the invention whole cells, preferably from a mammal, or extracts from such cells or extracts from subcellular compartments of such cells, are provided and contacted with a Class II HDAC-specific substrate, wherein deacetylation of the substrate by the Class II HDAC family or one or more members thereof generates a detectable reporter molecule. A first aliquot of the cells, or said extracts, is further contacted with the candidate Class II HDAC-specific inhibitor or candidate inhibitor of one or more member thereof and a second aliquot of the cells, or said extracts, is not. The quantity of the detectable reporter molecule is then measured for the first and second aliquots and the quantity of protein deacetylase activity for each aliquot is compared. In preferred embodiments, the quantity of the detectable reporter molecule is measured against a control standard for the protein deacetylase family or the one or more members thereof. In preferred embodiments, the substrate is cell-permeable.

A “candidate Class II HDAC-specific inhibitor” is an inhibitor of protein deacetylase which is to be tested for its ability to inhibit one or more members of the Class II HDAC family. A “Class II HDAC-specific substrate” is a substrate for one or more members of the Class II HDAC family. All other definitions are as described above.

In a seventh aspect, the invention provides a method for assessing isotype-specific activity of a candidate inhibitor of a member of the Class II HDAC family in whole cells ex vivo, in extracts of such cells, or in extracts of sub-cellular compartments of such cells. In the method according to this aspect of the invention whole cells, preferably from a mammal, or extracts from such cells or extracts from subcellular compartments of such cells, are provided and contacted with a Class II HDAC-specific substrate or an isotype-specific substrate for one or more member of the Class II HDAC family, wherein deacetylation of the substrate by the protein deacetylase generates a detectable reporter molecule. A first aliquot of the cells, or said extracts, is further contacted with the candidate isotype-specific inhibitor of the member of the Class II HDAC family and a second aliquot of the cells, or said extracts, is not. The quantity of the detectable reporter molecule is then measured for the first and second aliquots and the quantity of the detectable reporter molecule for each aliquot is compared. In preferred embodiments, the quantity of the detectable reporter molecule is measured against a control standard for the protein deacetylase family or the one or more member thereof. In preferred embodiments, the substrate is cell-permeable.

“Isotype-specific activity of a candidate inhibitor” is a determination of whether an inhibitor of protein deacetylation is specific for one or more member, but less than all members of the Class II HDAC family. All other definitions are as described above.

In an eighth aspect, the invention provides a method for assessing the efficacy of a Class II HDAC-specific inhibitor or an inhibitor of one or more member thereof in vivo. In the method according to this aspect of the invention, whole cells are provided, from a mammal. The cells are contacted with a cell permeable Class II HDAC-specific substrate or a cell permeable isotype specific substrate, wherein deacetylation of the substrate by the Class II HDAC family or one or more members thereof generates a detectable reporter molecule. The quantity of the reporter molecule is then determined. In preferred embodiments, the quantity is standardized against a known activity of the Class II HDACor one or more member thereof. Next, the mammal is administered the Class II HDAC-specific inhibitor or the inhibitor or one or more member thereof. After an appropriate period of time, whole cells are again taken from the mammal and contacted with the Class II HDAC-specific substrate. Next the quantity of the reporter molecule is determined. In preferred embodiments, the quantity is standardized against a known activity of the Class II HDAC or one or more members thereof. Then the quantity of the reporter molecule after administration of the Class II HDAC-specific inhibitor, or inhibitor of one or more member thereof, is compared with the quantity of the reporter molecule before administration of the Class II HDAC-specific inhibitor, or inhibitor of one or more member thereof. Significant decrease in the quantity of the reporter molecule after administration of the Class II HDAC-specific inhibitor, or inhibitor of one or more member thereof, is taken as a measure of efficacy.

Administration of the Class II HDAC-specific inhibitor may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the Class II HDAC family. In certain preferred embodiments, the bodily fluid obtained before administration of a HDAC-specific inhibitor is saved and quantification of the detectable reporter molecule in bodily fluids obtained before and after administration may be done simultaneously or nearly simultaneously.

All other definitions are as described above.

In a ninth aspect, the invention provides a method for assessing the efficacy and specificity of an isotype-specific inhibitor of one or more member of the Class II HDAC family in vivo. In the method according to this aspect of the invention, whole cells are provided from a mammal. The cells are contacted with a cell permeable isotype-specific substrate for the one or more member of the Class II HDAC family, wherein deacetylation of the substrate by the HDAC generates a detectable reporter molecule. The quantity of the reporter molecule is then determined. In preferred embodiments, the quantity is standardized against a known activity of the one or more member of the Class II HDAC family. Next, the mammal is administered the isotype-specific inhibitor. After an appropriate period of time, whole cells are again taken from the mammal and contacted with the isotype-specific substrate. Next the quantity of the reporter molecule is determined. In preferred embodiments, the quantity is standardized against a known activity of the one or more member of the Class II HDAC family. Then the quantity of the reporter molecule after administration of the isotype-specific inhibitor is compared with the quantity of the reporter molecule before administration of the isotype-specific inhibitor. Significant decrease in the quantity of the reporter molecule after administration of the isotype-specific inhibitor is taken as a measure of efficacy.

Administration of the isotype-specific inhibitor may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the member of Class II HDAC family. In certain preferred embodiments, the bodily fluid obtained before administration of the isotype-specific inhibitor is saved and quantification of the detectable reporter molecule in bodily fluids obtained before and after administration may be done simultaneously or nearly simultaneously.

All other definitions are as described above.

In a tenth aspect, the invention provides a method for assessing the efficacy of a Class II HDAC-specific inhibitor or an inhibitor of one or more member thereof in vivo by measuring the quantity of a detectable reporter molecule in bodily fluids from a mammal. In the method according to this aspect of the invention, the mammal is administered a cell-permeable Class II HDAC-specific substrate or an isotype-specific substrate, wherein deacetylation of the Class II HDAC-specific substrate or isotype-specific substrate generates a detectable reporter molecule. Bodily fluids from the mammal are obtained and the quantity of the detectable reporter molecule in the bodily fluids is determined. The mammal is then administered the Class II HDAC-specific inhibitor, or inhibitor of one or more member thereof, and after an appropriate time period the mammal is administered the Class II HDAC-specific substrate. Bodily fluids from the mammal are obtained and the quantity of the detectable reporter molecule in the bodily fluids is determined. The quantity of detectable reporter molecule in bodily fluids obtained prior to administration of the Class II HDAC-specific inhibitor, or inhibitor of one or more member thereof, is then compared with the quantity of the detectable reporter molecule in bodily fluids after administration of the Class II HDAC-specific inhibitor, or inhibitor of one or more member thereof. Significant decrease in the quantity of the reporter molecule after administration of the inhibitor is taken as a measure of efficacy.

Administration of the Class II HDAC-specific substrate or isotype-specific substrate and the Class II HDAC-specific inhibitor may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the one or more member of the Class II HDAC family. In certain preferred embodiments, the bodily fluid obtained before administration of the pan-inhibitor is saved and quantification of the detectable reporter molecule in bodily fluids obtained before and after administration may be done simultaneously or nearly simultaneously.

All other definitions are as described above.

In an eleventh aspect, the invention provides a method for assessing the efficacy of an isotype-specific inhibitor of one or more member of the Class II HDAC family in mammals in vivo by measuring the quantity of a detectable reporter molecule in bodily fluids. In the method according to this aspect of the invention, the mammal is administered a cell-permeable isotype-specific substrate for the one or more member of the Class II HDAC family, wherein deacetylation of the isotype-specific substrate generates the detectable reporter molecule. Bodily fluids from the mammal are obtained and the quantity of the detectable reporter molecule in the bodily fluids is determined. The mammal is then administered an isotype-specific inhibitor of one or more member of the Class II HDAC family and after an appropriate time period the mammal is administered the isotype-specific substrate. Bodily fluids from the mammal are obtained and the quantity of the detectable reporter molecule in the bodily fluids is determined. The quantity of detectable reporter molecule in bodily fluids obtained prior to administration of the isotype-specific inhibitor is then compared with the quantity of the detectable reporter molecule in bodily fluids after administration of the isotype-specific inhibitor. Significant decrease in the quantity of the reporter molecule after administration of the isotype-specific inhibitor is taken as a measure of efficacy.

Administration of the isotype-specific substrate and the isotype-specific inhibitor may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the one or more member of the Class II HDAC family. In certain preferred embodiments, the bodily fluid obtained before administration of the isotype-specific inhibitor is saved and quantification of the detectable reporter molecule in bodily fluids obtained before and after administration may be done simultaneously or nearly simultaneously. The detectable reporter molecule is capable of diffusing out of the cells and into bodily fluids.

All other definitions are as described above.

In a twelfth aspect, the invention provides a method for assessing the efficacy of a Class II HDAC-specific activator or an activator of one or more member thereof in vivo. In the method according to this aspect of the invention, whole cells are provided from a mammal. The cells are contacted with a cell permeable Class II HDAC-specific substrate or a cell permeable isotype specific substrate, wherein deacetylation of the substrate by the Class II HDAC family or one or more members thereof generates a detectable reporter molecule. The quantity of the reporter molecule is then determined. In preferred embodiments, the quantity is standardized against a known activity of the Class II HDAC family or the one or more members thereof. Next, the mammal is administered the Class II HDAC-specific activator, or the activator of one or more member thereof. After an appropriate period of time, whole cells are again taken from the mammal and contacted with the Class II HDAC-specific substrate, or isotype specific substrate. Next the quantity of the reporter molecule in the whole cells is determined. In preferred embodiments, the quantity is standardized against a known activity of the Class II HDAC family or the one or more members thereof. Then the quantity of the reporter molecule after administration of the Class II HDAC-specific activator, or activator of one or more member thereof, is compared with the quantity of the reporter molecule before administration of the Class II HDAC-specific activator, or activator of one or more member thereof. Significant increase in the quantity of the reporter molecule after administration of the Class II HDAC-specific activator, or activator of one or more member thereof, is taken as a measure of efficacy.

A Class II HDAC-specific activator or an activator of one or more member thereof is a molecule that activates at least one, and up to all members of the protein deacetylase family.

Administration of the activator may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the one or more member of the Class II HDAC family. In certain preferred embodiments, the bodily fluid obtained before administration of the activator is saved and quantification of the detectable reporter molecule in bodily fluids obtained before and after administration may be done simultaneously or nearly simultaneously. The detectable reporter molecule is capable of diffusing out of the cells and into bodily fluids.

All other definitions are as described above.

In a thirteenth aspect, the invention provides a method for assessing the efficacy and specificity of an isotype-specific activator of one or more member of the Class II HDAC family in vivo. In the method according to this aspect of the invention, whole cells are provided from a mammal. The cells are contacted with a cell permeable isotype-specific substrate for the one or more member of the Class II HDAC family, wherein deacetylation of the substrate by the HDAC generates a detectable reporter molecule. The quantity of the reporter molecule is then determined. In preferred embodiments, the quantity is standardized against a known activity of the one or more member of the Class II HDAC family. Next, the mammal is administered the isotype-specific activator. After an appropriate period of time, whole cells are again taken from the mammal and contacted with the isotype-specific substrate. Next the quantity of the reporter molecule is determined. In preferred embodiments, the quantity is standardized against a known activity of the one or more member of the Class II HDAC family. Then the quantity of the reporter molecule after administration of the isotype-specific activator is compared with the quantity of the reporter molecule before administration of the isotype-specific activator. Significant increase in the quantity of the reporter molecule after administration of the isotype-specific activator is taken as a measure of efficacy.

An isotype-specific activator of one or more member of a protein deacetylase family is a molecule that increases the activity and/or quantity of one or more member, but not all members of the protein deacetylase family. All other definitions are as described above.

Administration of the isotype-specific activator may be by any acceptable route, including without limitation oral, parenteral, sublingual, intravenous, intraocular, topical, intranasal, intraventricular, intravesicular and intrarectal. Bodily fluids include, without limitation blood, plasma, sputum, urine and cerebrospinal fluid. In certain preferred embodiments, each quantitation of the detectable reporter molecule is standardized against a known activity of the HDAC. In certain preferred embodiments, the bodily fluid obtained before administration of the isotype-specific activator is saved and quantification of the detectable reporter molecule in bodily fluids obtained before and after administration may be done simultaneously or nearly simultaneously. The detectable reporter molecule is capable of diffusing out of the cells and into bodily fluids.

All other definitions are as described above.

In a fourteenth aspect, the invention provides a method for assessing the efficacy of a Class II HDAC-specific activator or an activator of one or more members thereof in vivo by measuring the quantity of a detectable reporter molecule in bodily fluids from a mammal. In the method according to this aspect of the invention, the mammal is administered a cell-permeable Class II HDAC-specific substrate or substrate for one or more members thereof, wherein deacetylation of the Class II HDAC-specific substrate or isotype-specific substrate generates the detectable reporter molecule. Bodily fluids from the mammal are obtained and the quantity of the detectable reporter molecule in the bodily fluids is determined. In preferred embodiments, the quantity is standardized against a known activity of the one or more members of the Class II HDAC family. The mammal is then administered the Class II HDAC-specific activator, or activator of one or more members thereof, and after an appropriate time period the mammal is administered the Class II HDAC-specific substrate or isotype specific substrate. Bodily fluids from the mammal are obtained and the quantity of the detectable reporter molecule in the bodily fluids is determined. In preferred embodiments, the quantity is standardized against a known activity of the one or more members of the Class II HDAC family. The quantity of detectable reporter molecule in bodily fluids obtained prior to administration of the Class II HDAC-specific activator, or activator of one or more member thereof, is then compared with the quantity of the detectable reporter molecule in bodily fluids after administration of the Class II HDAC-specific activator, or activator of one or more member thereof. Significant increase in the quantity of the reporter molecule after administration of the Class II HDAC-specific activator, or activator of one or more member thereof, is taken as a measure of efficacy.

A “class II HDAC-specific activator” is a molecule that activates at least one, and up to all Class II HDACs, but does not activate Class I HDACs to a comparable extent.

All other definitions are as described above.

In a fifteenth aspect, the invention provides a method for assessing the efficacy of an isotype-specific activator of one or more member of the Class II HDAC family in a mammal in vivo by measuring the quantity of a detectable reporter molecule in bodily fluids from the mammal. In the method according to this aspect of the invention, the mammal is administered a cell-permeable isotype-specific substrate for a Class II HDAC, or one or more member thereof, wherein deacetylation of the isotype-specific substrate generates the detectable reporter molecule. Bodily fluids from the mammal are obtained and the quantity of the detectable reporter molecule in the bodily fluids is determined. In preferred embodiments, the quantity is standardized against a known activity of the one or more member of the Class II HDAC family. The mammal is then administered an isotype-specific activator of one or more member of the Class II HDAC family and after an appropriate time period the mammal is administered the isotype-specific substrate. Bodily fluids from the mammal are obtained and the quantity of the detectable reporter molecule in the bodily fluids is determined. In preferred embodiments, the quantity is standardized against a known activity of the one or more member of the Class II HDAC family. The quantity of detectable reporter molecule in bodily fluids obtained prior to administration of the isotype-specific activator is then compared with the quantity of the detectable reporter molecule in bodily fluids after administration of the isotype-specific activator. Significant increase in the quantity of the reporter molecule after administration of the isotype-specific activator is taken as a measure of efficacy.

An “isotype-specific activator of one or more Class II HDACs” is a molecule that activates at least one, but less than all Class II HDACs, but does not activate Class I HDACs to a comparable extent.

All other definitions are as described above.

In a sixteenth aspect, the invention provides a method for assessing the activity of a candidate Class II HDAC-specific activator or an activator of one or more members thereof in whole cells ex vivo. In the method according to this aspect of the invention whole cells from a mammal are provided and contacted with a cell-permeable a Class II HDAC-specific substrate or an isotype-specific substrate, wherein deacetylation of the substrate by the Class II HDAC family or one or more members thereof generates a detectable reporter molecule. A first aliquot of the cells is further contacted with a candidate Class II HDAC-specific activator, or an activator of one or more member thereof, and a second aliquot of the cells is not. The quantity of the detectable reporter molecule is then measured for the first and second aliquots and the quantity of Class II HDAC-specific activity, or activity of one or more member thereof, for each aliquot is compared. In preferred embodiments, the quantity of the detectable reporter molecule is measured against a control standard for the protein deacetylase family or the one or more members thereof.

In preferred embodiments, the quantity of the detectable reporter molecule is measured against a control standard for the Class II HDAC family or the one or more members thereof.

All other definitions are as described above.

In a seventeenth aspect, the invention provides a method for assessing the activity of a candidate isotype-specific activator of a member of the Class II HDAC family ex vivo. In the method according to this aspect of the invention whole cells from a mammal are provided and contacted with a cell-permeable isotype-specific substrate, wherein deacetylation of the substrate by the member of the Class II HDAC family generates a detectable reporter molecule. A first aliquot of the cells is further contacted with a candidate isotype-specific activator of the member of the Class II HDAC family and a second aliquot of the cells is not. The quantity of the detectable reporter molecule is then measured for the first and second aliquots and the quantity of isotype-specific HDAC activity for each aliquot is compared. In preferred embodiments, the quantity of the detectable reporter molecule is measured against a control standard for the member of the Class II HDAC family.

All definitions are as described above.

In an eighteenth aspect, the invention provides compounds of formula (I), with the proviso that the compound is not Boc-Lys(Ac)-AMC or Boc-Lys(Tfa)-AMC:

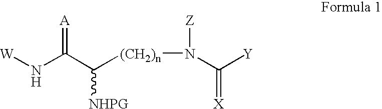

wherein
X is selected from the group consisting of O, S, NH and N(alkyl);
Y is selected from the group consisting of OH, alkoxy, alkyl, alkenyl and alkynyl, each of which alkoxy, alkyl, alkenyl and alkynyl, is optionally substituted with 1 to 7 substituents independently selected from the group consisting of halo, cyano, alkoxy, alkylamino and alkylthio;
Z is selected from the group consisting of H, alkyl, alkenyl and alkynyl;
n is an integer ranging from 0 to 12;
PG is a protecting group (preferably selected from the group consisting of MeCO, CF₃CO—, Boc and CBZ), an amino acide or a peptide;
A is selected from the group consisting of O, S, NH and N(alkyl); and
W is a carboxycylic, heterocyclic, saturated or unsaturated, aromatic or heteroaromatic mono-, bi-, tri- or tetracyclic ring system (preferably a mono- or bicyclic aryl or a mono- or bicyclic heteroaryl ring system).

In another aspect, the invention provides for the use of a compound according to Formula I as a substrate for Class II histone deacetylases.

In another aspect, the invention provides a complex of a compound according to Formula I bound to a Class II histone deacetylase.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-heteroaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, ethenyl, ethynyl, propyl, propenyl, propynyl, and cyclopropyl.

The term “alkyl” is intended to mean a straight or branched chain aliphatic group having from 1 to 12 carbon atoms, preferably 1-8 carbon atoms, and more preferably 1-6 carbon atoms, and more preferably 1-4, most preferably 4 carbon atoms. Other preferred alkyl groups have from 2 to 12 carbon atoms, preferably 2-8 carbon atoms and more preferably 2-6 carbon atoms and more preferably 2-4 carbons atoms. Preferred alkyl groups include, without limitation, methyl, ethyl, propyl, isopropyl, butyl, isobutyl, sec-butyl, tert-butyl, pentyl, and hexyl. A “C₀” alkyl (as in “C₀-C₃-alkyl”) is a covalent bond.

The term “alkenyl” is intended to mean an unsaturated straight or branched chain aliphatic group with one or more carbon-carbon double bonds, having from 2 to 12 carbon atoms, preferably 2-8 carbon atoms, and more preferably 2-6 carbon atoms. Preferred alkenyl groups include, without limitation, ethenyl, propenyl, butenyl, pentenyl, and hexenyl.

The term “alkynyl” is intended to mean an unsaturated straight or branched chain aliphatic group with one or more carbon-carbon triple bonds, having from 2 to 12 carbon atoms, preferably 2-8 carbon atoms, and more preferably 2-6 carbon atoms. Preferred alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “cycloalkyl” is intended to mean a saturated or unsaturated mono-, bi, tri- or poly-cyclic hydrocarbon group having about 3 to 15 carbons, preferably having 3 to 12 carbons, preferably 3 to 8 carbons, and more preferably 3 to 6 carbons. In certain preferred embodiments, the cycloalkyl group is fused to an aryl, heteroaryl or heterocyclic group. Preferred cycloalkyl groups include, without limitation, cyclopenten-2-enone, cyclopenten-2-enol, cyclohex-2-enone, cyclohex-2-enol, cyclopropyl, cyclobutyl, cyclopentyl, cyclopentenyl, cyclohexyl, cyclohexenyl, cycloheptyl, and cyclooctyl.

The term “heteroalkyl” is intended to mean a saturated or unsaturated, straight or branched chain aliphatic group, wherein one or more carbon atoms in the chain are independently replaced by a heteroatom selected from the group consisting of O, S, and N.

The term “aryl” is intended to mean a mono-, bi-, tri- or polycyclic C₆-C₁₄ aromatic moiety, preferably comprising one to three aromatic rings. Preferably, the aryl group is a C₆-C₁₀ aryl group, more preferably a C₆ aryl group. Preferred aryl groups include, without limitation, phenyl, naphthyl, anthracenyl, and fluorenyl.

The terms “aralkyl” or “arylalkyl” is intended to mean a group comprising an aryl group covalently linked to an alkyl group. If an aralkyl group is described as “optionally substituted”, it is intended that either or both of the aryl and alkyl moieties may independently be optionally substituted or unsubstituted. Preferably, the aralkyl group is (C₁-C₆)alk(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is a mono-, bi-, or polycyclic structure having from about 3 to about 14 atoms, wherein one or more atoms are independently selected from the group consisting of N, O, and S. The ring structure may be saturated, unsaturated or partially unsaturated. In certain preferred embodiments, the heterocyclic group is non-aromatic. In a bicyclic or polycyclic structure, one or more rings may be aromatic; for example one ring of a bicyclic heterocycle or one or two rings of a tricyclic heterocycle may be aromatic, as in indan and 9,10-dihydro anthracene. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” is intended to mean a mono-, bi-, tri- or polycyclic group having 5 to 14 ring atoms, preferably 5, 6, 9, or 10 ring atoms; having 6, 10, or 14 pi electrons shared in a cyclic array; and having, in addition to carbon atoms, between one or more heteroatoms independently selected from the group consisting of N, O, and S. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

Preferred heterocyclyls and heteroaryls include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thianthrenyl, thiazolyl, thienyl, thienothiazolyl, thienooxazolyl, thienoimidazolyl, thiophenyl, triazinyl, triazolyl (e.g., 1,2,3-triazolyl, 1,2,4-triazolyl, 1,2,5-triazolyl, 1,3,4-triazolyl), and xanthenyl.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise), are:
(a) halo, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈alkyl, C₁-C₈alkenyl, C₁-C₈alkoxy, C₁-C₈alkoxycarbonyl, aryloxycarbonyl, C₂-C₈acyl, C₂-C₈acylamino, C₁-C₈alkylthio, arylalkylthio, arylthio, C₁-C₈alkylsulfinyl, arylalkylsulfinyl, arylsulfinyl, C₁-C₈alkylsulfonyl, arylalkylsulfonyl, arylsulfonyl, C₀-C₆N-alkylcarbamoyl, C₂-C₁₅N,N-dialkylcarbamoyl, C₃-C₇cycloalkyl, aroyl, aryloxy, arylalkyl ether, aryl, aryl fused to a cycloalkyl or heterocycle or another aryl ring, C₃-C₇heterocycle, C₅-C₁₅heteroaryl or any of these rings fused or spiro-fused to a cycloalkyl, heterocyclyl, or aryl, wherein each of the foregoing is further optionally substituted with one more moieties listed in (a), above; and
(c) —(CR³²R³³)ₛ—NR³⁰R³¹, wherein s is from 0 (in which case the nitrogen is directly bonded to the moiety that is substituted) to 6, R³² and R³³ are each independently hydrogen, halo, hydroxyl or C₁-C₄alkyl, and R³⁰ and R³¹ are each independently hydrogen, cyano, oxo, hydroxyl, —C₁-C₈alkyl, C₁-C₈heteroalkyl, C₁-C₈alkenyl, carboxamido, C₁-C₃alkyl-carboxamido, carboxamido-C₁-C₃alkyl, amidino, C₂-C₈hydroxyalkyl, C₁-C₃alkylaryl, aryl-C₁-C₃alkyl, C₁-C₃alkylheteroaryl, heteroaryl-C₁-C₃alkyl, C₁-C₃alkylheterocyclyl, heterocyclyl-C₁-C₃alkyl C₁-C₃alkylcycloalkyl, cycloalkyl-C₁-C₃alkyl, C₂-C₈alkoxy, C₂-C₈alkoxy-C₁-C₄alkyl, C₁-C₈alkoxycarbonyl, aryloxycarbonyl, aryl-C₁-C₃alkoxycarbonyl, heteroaryloxycarbonyl, heteroaryl-C₁-C₃alkoxycarbonyl, C₁-C₈acyl, C₀-C₈alkyl-carbonyl, aryl-C₀-C₈alkyl-carbonyl, heteroaryl-C₀-C₈alkyl-carbonyl, cycloalkyl-C₀-C₈alkyl-carbonyl, C₀-C₈alkyl-NH-carbonyl, aryl-C₀-C₈alkyl-NH-carbonyl, heteroaryl-C₀-C₈alkyl-NH-carbonyl, cycloalkyl-C₀-C₈alkyl-NH-carbonyl, C₀-C₈alkyl-O-carbonyl, aryl-C₀-C₈alkyl-O-carbonyl, heteroaryl-C₀-C₈alkyl-O-carbonyl, cycloalkyl-C₀-C₈alkyl-O-carbonyl, C₁-C₈alkylsulfonyl, arylalkylsulfonyl, arylsulfonyl, heteroarylalkylsulfonyl, heteroarylsulfonyl, C₁-C₈alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, or protecting group, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰—Y³¹—), wherein said heterocyclyl may also be bridged (forming a bicyclic moiety with a methylene, ethylene or propylene bridge); wherein
   X³⁰ is selected from the group consisting of C₁-C₈alkyl, C₂-C₈alkenyl-, C₂-C₈alkyl-, —C₀-C₃alkyl-C₂-C₈alkenyl-C₀-C₃alkyl, C₀-C₃alkyl-C₂-C₈alkynyl-C₀-C₃alkyl, C₀-C₃alkyl-O—C₀-C₃alkyl-, HO—C₀-C₃alkyl-, C₀-C₄alkyl-N(R³⁰)—C₀-C₃alkyl-, N(R³⁰)(R³¹)—C₀-C₃alkyl-, N(R³⁰)(R³¹)—C₀-C₃alkenyl-, N(R³⁰)(R³¹)—C₀-C₃alkynyl-, (N(R³⁰)(R³¹))₂—C═N—, C₀-C₃alkyl-S(O)₀₋₂—C₀-C₃alkyl-, CF₃—C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocycyl are optionally substituted with from 1 to 3 substituents from (a); and Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)—N(R³¹)—, —N(R³⁰)—C(NR³⁰)—N(R³¹)—, —N(R³⁰)—C(NR³¹)—, —C(NR³¹)—N(R³⁰), —N(R³⁰)—C(S)—N(R³¹)—, —N(R³⁰)—C(O)—O—, —O—C(O)—N(R³¹)—, —N(R³⁰)—C(S)—O—, —O—C(S)—N(R³¹)—, —S(O)₀₋₂—, —SO₂N(R³¹)—, —N(R³¹)—SO₂— and —N(R³⁰)—SO₂N(R³¹)—.

As a non-limiting example, substituted phenyls include 2-fluorophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl —CO—.

When there are two optional substituents bonded to adjacent atoms of a ring structure, such as for example phenyl, thiophenyl, or pyridinyl, the substituents, together with the atoms to which they are bonded, optionally form a 5- or 6-membered cycloalkyl or heterocycle having 1, 2, or 3 annular heteroatoms.

In a preferred embodiment, hydrocarbyl, heteroalkyl, heterocyclic, aryl, groups are unsubstituted.

In other preferred embodiments, hydrocarbyl, heteroalkyl, heterocyclic, aryl, groups are substituted with from 1 to 3 independently selected substituents.

In a preferred embodiment, a heterocyclic group is substituted on carbon, nitrogen and/or sulfur at one or more positions. Preferred substituents on nitrogen include, but are not limited to alkyl, aryl, aralkyl, alkylcarbonyl, alkylsulfonyl, arylcarbonyl, arylsulfonyl, alkoxycarbonyl, or aralkoxycarbonyl. Preferred substituents on sulfur include, but are not limited to, oxo and C₁₋₆alkyl.

The term “halogen” or “halo” is intended to mean chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

The term “radical” is intended to mean a chemical moiety comprising one or more unpaired electrons.

Where optional substituents are chosen from “one or more” groups it is to be understood that this definition includes all substituents being chosen from one of the specified groups or the substituents being chosen from two or more of the specified groups.

In addition, substituents on cyclic moieties (i.e., cycloalkyl, heterocyclyl, aryl, heteroaryl) include 5-6 membered mono- and 9-14 membered bi-cyclic moieties fused to the parent cyclic moiety to form a bi- or tri-cyclic fused ring system. Substituents on cyclic moieties also include 5-6 membered mono- and 9-14 membered bi-cyclic moieties attached to the parent cyclic moiety by a covalent bond to form a bi- or tri-cyclic bi-ring system. For example, an optionally substituted phenyl includes, but is not limited to, the following:

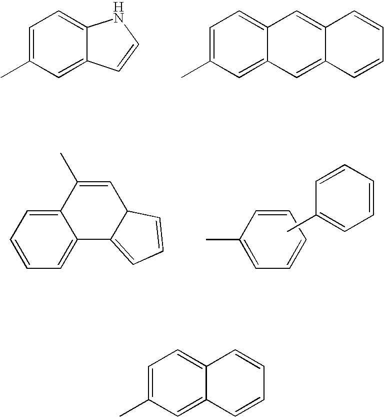

An “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides.

The term “protecting group” is intended to mean a group used in synthesis to temporarily mask the characteristic chemistry of a functional group because it interferes with another reaction. A good protecting group should be easy to put on, easy to remove and in high yielding reactions, and inert to the conditions of the reaction required. A protecting group or protective group is introduced into a molecule by chemical modification of a functional group in order to obtain chemoselectivity in a subsequent chemical reaction. One skilled in the art will recognize that during any of the processes for preparation of the compounds in the present invention, it may be necessary and/or desirable to protect sensitive or reactive groups on any of the molecules concerned. This may be achieved by means of conventional protecting groups, such as but not limited to Bn- (or —CH₂Ph), —CHPh₂, alloc (or CH₂═CH—CH₂—O—C(O)—), BOC—, —Cbz (or Z-), —F-moc, —C(O)—CF₃, N-Phthalimide, 1-Adoc-, TBDMS-, TBDPS-, TMS-, TIPS-, IPDMS-, —SiR₃, SEM-, t-Bu-, Tr-, THP- and Allyl-. These protecting groups may be removed at a convenient stage using methods known from the art.

An “amino acid residue” refers to any residue of a natural or unnatural amino acid, non-limiting examples of which are residues of alanine, arginine, asparagine, aspartic acid, cysteine, homocysteine, glutamine, glutamic acid, isoleucine, norleucine, glycine, phenylglycine, leucine, histidine, methionine, lysine, phenylalanine, homophenylalanine, ornithine, praline, serine, homoserine, valine, norvaline, threonine, tryptophane, tyrosine and the like. With the exception of glycine, all amino acids may be in the D-, L- or D,L-form.

In another aspect, the invention provides a method for assessing efficacy and specificity of a candidate HDAC inhibitor in vitro. In the method according to this aspect of the invention one or more individual Class II HDAC isotype-specific enzymes are provided and contacted with an appropriate Class II HDAC-specific substrate or an isotype-specific substrate for one or more member of the Class II HDAC families, wherein deacetylation of the substrate by the protein deacetylase generates a detectable reporter molecule. A first aliquot of each enzyme is further contacted with the candidate inhibitor and a second aliquot of the enzyme is not. The quantity of the detectable reporter molecule is then measured for the first and second aliquots and the quantity of the detectable reporter molecule for each aliquot is compared. In preferred embodiments, the quantity of the detectable reporter molecule is measured against a control standard for the protein deacetylase family or the one or more member thereof. Significant decrease in the quantity of the reporter molecule after contact of an enzyme with said candidate inhibitor is taken as a measure of efficacy. In a preferred embodiment, the inhibitor is an inhibitor of one or more HDAC enzymes. In another a preferred embodiment, the inhibitor is a Class-specific HDAC inhibitor.

In another aspect of the present invention, the invention provides an HDAC inhibitor identified by a method and/or use of a substrate as herein described.

One of skill in the art will realize that by combining a Class I HDAC-specific substrate and a Class II HDAC-specific substrate in a whole cell HDAC assay or cellular extract HDAC assay, total Class I and Class II HDAC activity of the cell or cellular extract can be determined. Similarly, use of individual Class-specific substrates can determine total Class-specific HDAC activity. Similarly, use of individual isotype-specific substrates or combinations of substrates each specific for a particular HDAC isotype or subset thereof can determine the activity of individual HDAC isotypes, or combinations of specific isotypes.

The present invention further provides for the use of a compound of formula (I) as a substrate for an appropriate Class II histone deacetylase family or one or more member thereof. The present invention further provides for the use of said substrates for screening candidate HDAC inhibitors or candidate HDAC activators to identify HDAC inhibitors or HDAC activators, respectively.

Because compounds of the invention are substrates for the Class II histone deacetylase families or one or more member thereof, they are useful research tools for in vitro study of histone deacetylases and their role in biological processes.

The following examples are intended to further illustrate certain particularly preferred embodiments of the invention and are not intended to limit the scope of the invention in any way.

### Example 1

### Intracellular and Excellular Deacetylase Activity of Human 293T Cells Using Boc-Lys(Ac)-AMC as Substrate

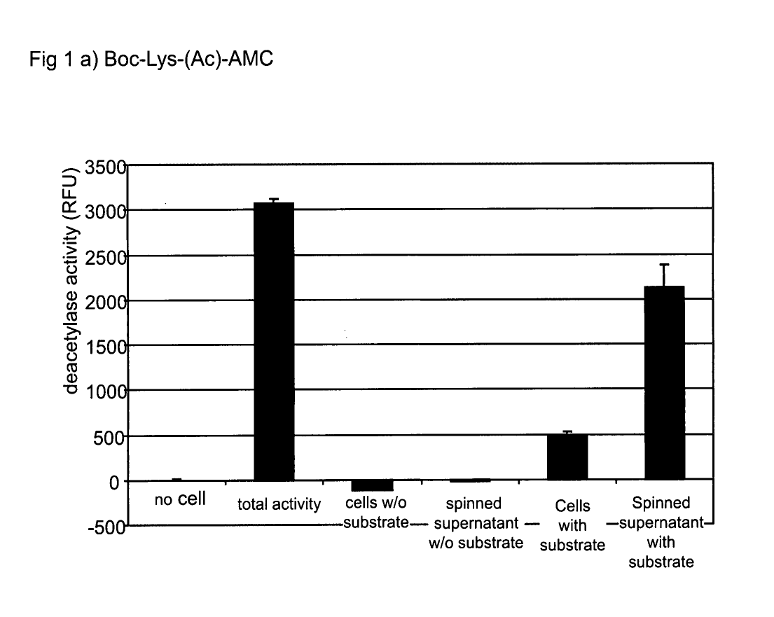

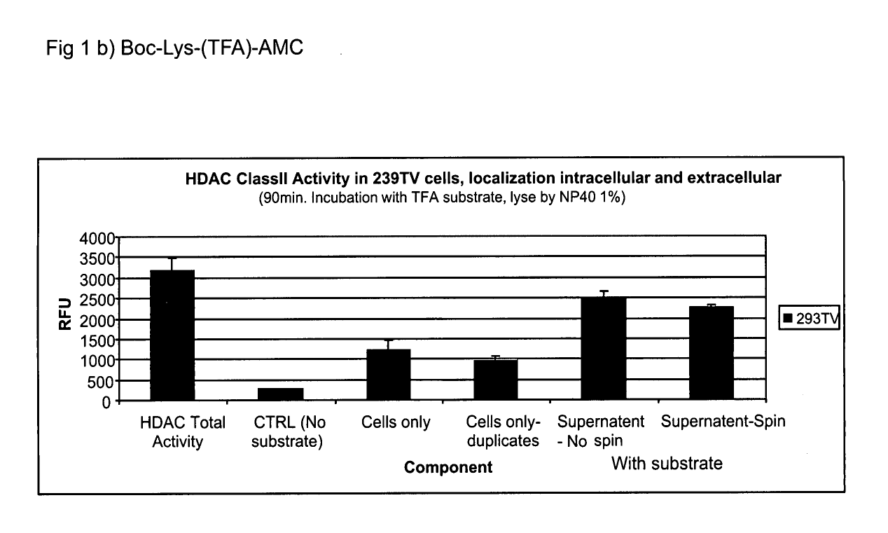

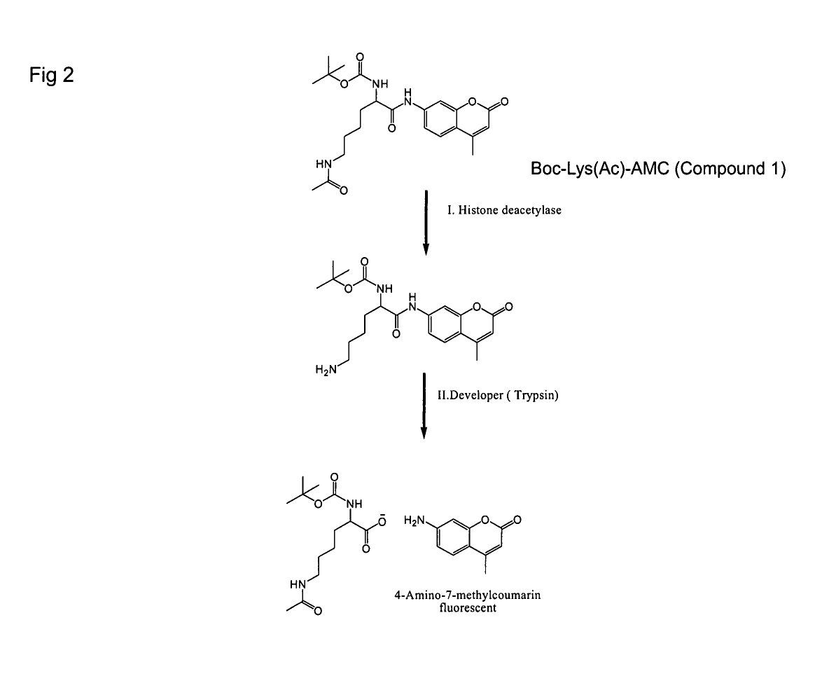

Freshly trypsinized cells (293T) were dispensed into 96-well black Costar E1A/RIA plates (Corning Inc., Corning, N.Y.). Small molecule substrate Boc-Lys(Ac)-AMC (Bachem Biosciences Inc., King of Prussia, Philadelphia) were added to cell suspension with the final concentration of 300 uM. Cells were placed in 37° C. incubator with 5% CO₂ for indicated time period. Supernatant was collected if necessary and subject to spinning. Reaction was stopped by adding a freshly prepared Fluor-de-Lys™ developer (Biomol, Plymouth Meeting, Philadelphia) with 1 uM TSA (Biomol, Plymouth Meeting, Philadelphia) in assay buffer (25 mM Tris, HCl pH8.0, 137 mM NaCl, 2.7 mM KCl, 1 mM MgCl₂) plus 1% NP-40 into supernatant or cell pellets. Fluorescence was developed for 15 minutes at 37° C. and read in a fluorometer (SPECTRAMAX GeminiXS, Molecular Devices, Sunnylvale, Calif.) with an excitation wavelength at 360 nm, emission at 470 nm, and a cutoff of 435 nm. As shown in FIG. 1, significant intracellular and excellular deacetylase activity could be detected, suggesting that Boc-Lys(Ac)-AMC could permeablize into cells and generated product (Boc-Lys-AMC) could be diffused into culture media and could be subsequently detected by developing fluorescence. In contrast, when there is no substrates added, neither supernatant from cultured cells nor cell pellets have HDAC activity. The flow chart of the assay is shown in FIG. 2.

### Example 2

### Whole Cell Activity in Human Cancer Cells and Normal Cells Using Boc-Lys(Ac)-AMC

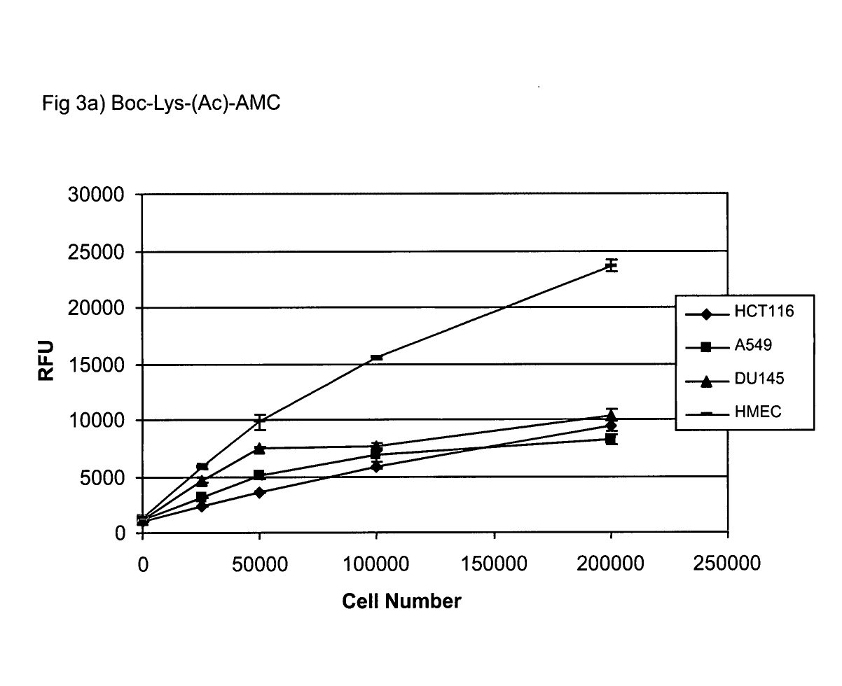

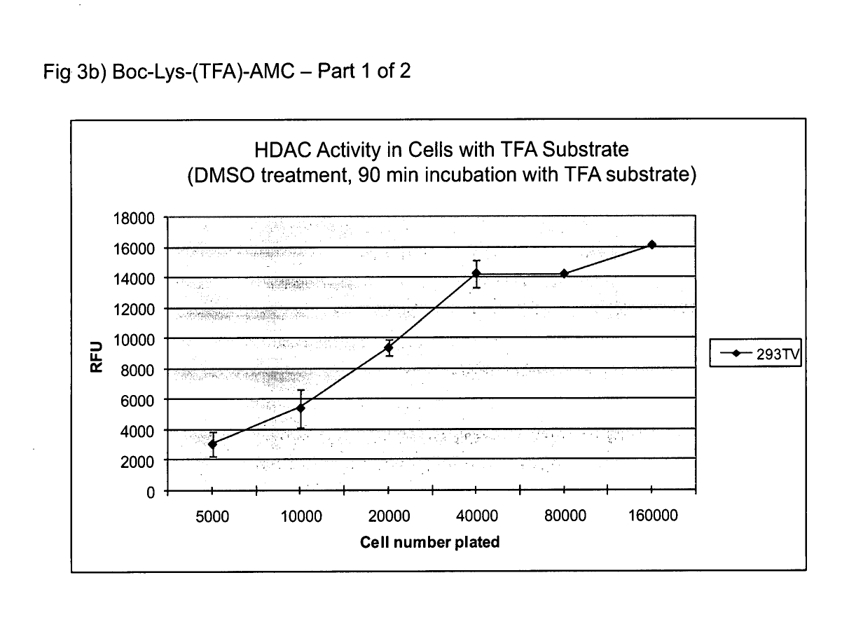

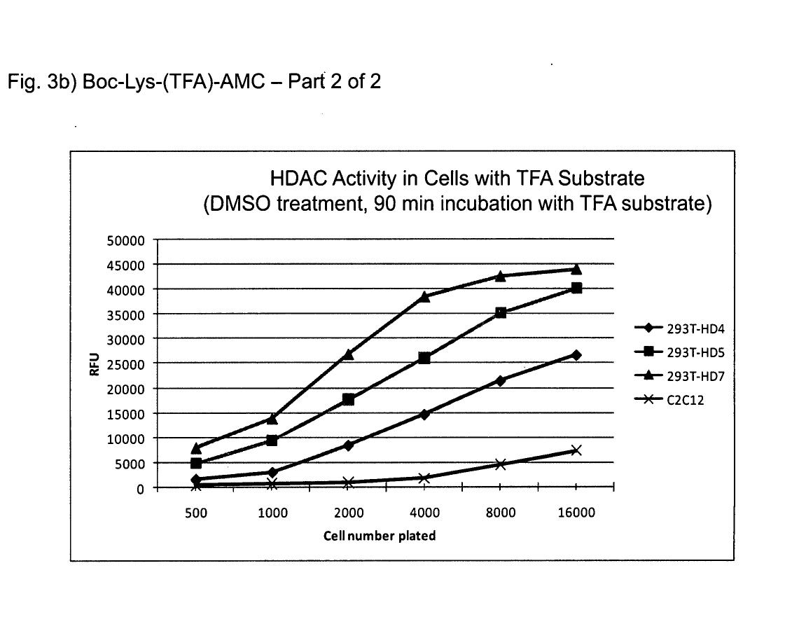

Freshly trypsinized cells were dispensed into 96-well black Costar E1A/RIA plates (Corning Inc., Corning, N.Y.). Small molecule substrate Boc-Lys(Ac)-AMC (Bachem Biosciences Inc., King of Prussia, Philadelphia) was added to cell suspension with the final concentration of 300 uM. Cells were placed in 37° C. incubator with 5% CO₂ for 90 minutes. Reaction was stopped by adding a freshly prepared Flouor-de-Lys™ deleveloper (Biomol, Plymouth Meeting, Philadelphia) with 1 uM TSA (Biomol) in assay buffer (25 mM Tris, HCl pH8.0, 137 mM NaCl, 2.7 mM KCl, 1 mM MgCl₂) plus 1% NP-40. With the presence of 1% NP-40, both excellular and intracellular HDAC activity was measured in cultured cells altogether. Fluorescence was developed for 15 minutes at 37 C and read in a fluorometer (SPECTRAMAX GeminiXS, Molecular Devices, Sunnylvale, Calif.) with an excitation wavelength at 360 nm, emission at 470 nm, and a cutoff of 435 nm. In cell lines we have tested (HCT116, A549, Du145, HMEC, 293T etc.), the total HDAC activity was a function of cell numbers (see FIG. 3).

### Example 3

### Effect of Boc-LysAc-AMC Substrate Concentration on Deacetylase Activity in Human Cancer Cell Lines

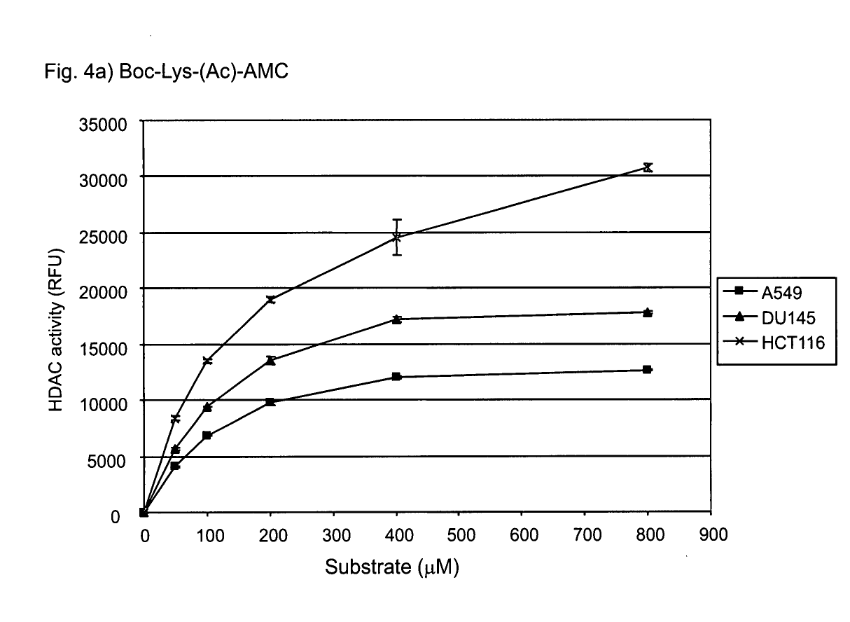

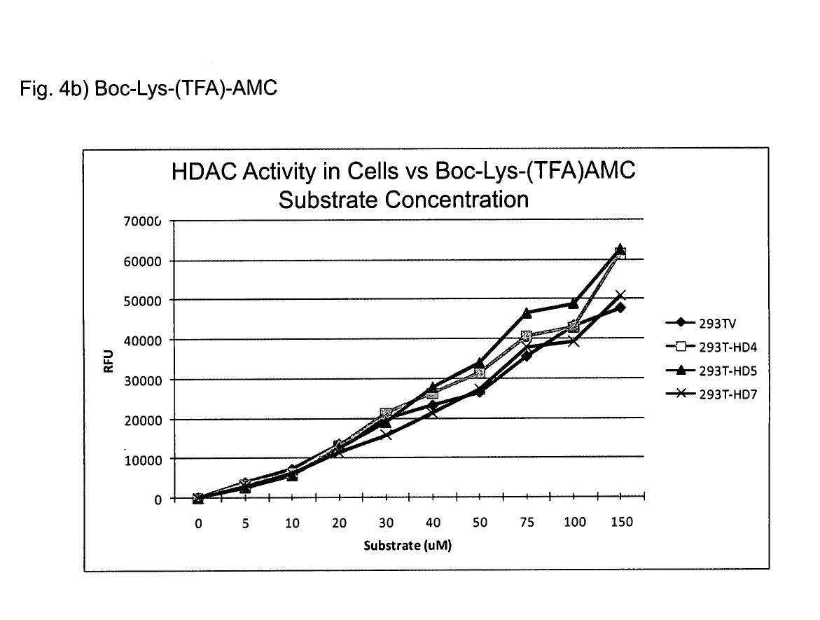

Cells were trypsinized and counted by trypan blue exclusion. Live cells (4×10⁴ A549 cells, or 1×10⁵ HCT116 cells, or 5×10⁴ Du145 cells) were distributed to each well of the 96-well plate. HDAC small molecule substrate Boc-Lys(Ac)-AMC with a range of final concentrations was added into cell suspensions and incubated with cells for 90 minutes at 37 C before reaction was stopped, and fluorescence was developed and read. As shown in FIG. 4, effect of substrate concentration on whole cell deacetylase activity was measured. Km of Boc-Lys(Ac)-AMC ranged from 150 μM to 220 μM.

### Example 4

### Activity of HDAC Pan or Isotype-Specific Inhibitors in Intact Cancer Cells Using Boc-Lys(Ac)-AMC as Substrate

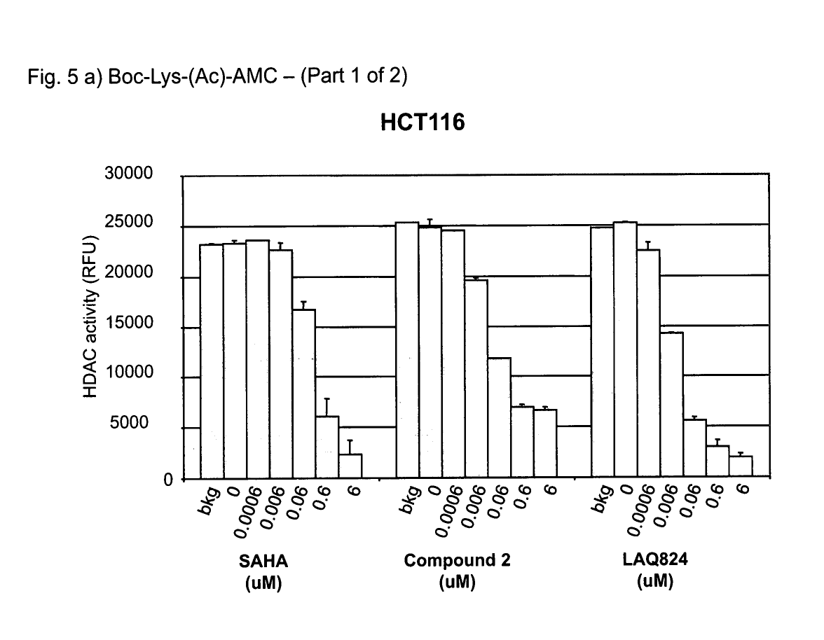

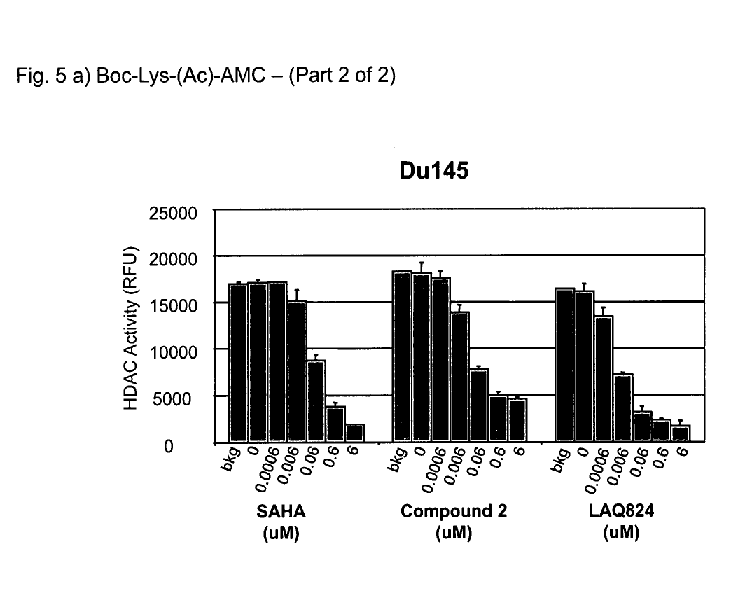

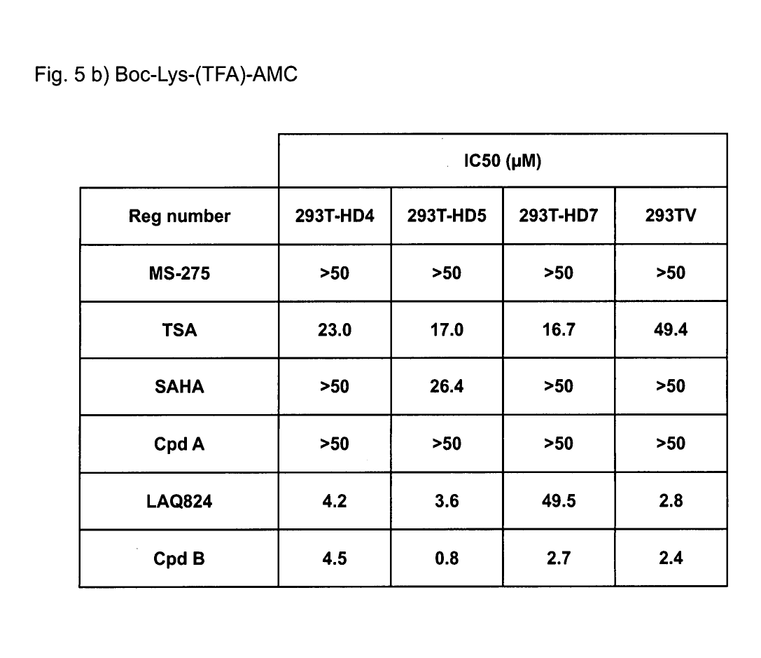

Human cancer cell lines (A549, Du145 and HCT116, 293T, Jurkat-T, Panc1) were treated with various concentrations of HDAC inhibitors for indicated time period before the enzyme substrate Boc-Lys(Ac)-AMC was added into cultured cells. Inhibitors could be pan-class I/II inhibitor (SAHA, LAQ-824) or isotype-specific class I inhibitors (against HD1, 2, 3), such as MS-275 or Compound 2. HDAC enzyme assay in intact cells was carried out as described in Example 2. The concentration which inhibits 50% of total HDAC activity (IC₅₀) in whole cells was determined by analyzing the dose-response curve of enzyme inhibition, as shown in FIG. 5 and Table 1.

TABLE 1
whole cell deacetylase IC50 of HDAC inhibitors or other
chemotherapeutic agents in various human cancer cells

| | IC50 (uM) | | | | | |
|---|---|---|---|---|---|---|
| | A549 | Du145 | HCT116 | 293T | Jurkat T | Panc-1 |
| Compound 2 | 0.4 | 0.6 | 0.4 | 0.5 | 0.2 | 0.2 |
| SAHA | 0.5 | 0.6 | 3 | 2 | 0.7 | 1 |
| MS-275 | 0.4 | 0.3 | 3 | 2 | 0.3 | 0.5 |
| LAQ-824 | 0.02 | 0.05 | 0.06 | 0.04 | 0.04 | nd |
| Taxol | | | | >50 | | |
| compound 4 | | | | >50 | | |

results are mean IC₅₀ from at least 2 independent experiments
cells were pre-incubated with inhibitors for 16 hours before reaction was stopped and read
compound 4 is a CDK2 inhibitor from BMS

### Example 5

### Whole Cell HDAC Activity of White Blood Cells Using Boc-Lys(Ac)-AMC as Substrate

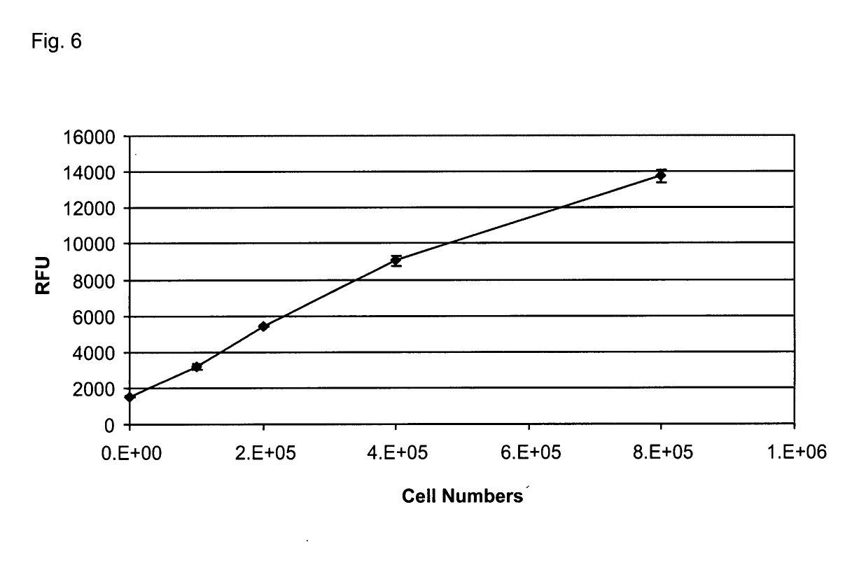

Whole blood (human or mouse) was centrifuged at 2500 rpm for 10 minutes at ambient temperature in a Sorvall RT-7 centrifuge (Mandel Scientific Co., Guelph, Ontario). Plasma was removed and buffy coat was collected. Five volumes of Erythrocyte Lysis Buffer (EL) (Qiagen Canada Inc., Mississauga, Ontario) were added to buffy coat. Buffy coat was incubated on ice for 20 minutes before it was centrifuged at 400 g for 10 minutes at 4° C. Supernatant was removed and buffy coat was washed twice with EL buffer and re-centrifugation. Buffy coat was resuspended in RPMI media and cells (white blood cells) were counted with trypan blue exclusion. White blood cells were plated into 96-well dish in RPMI plus 10% fetal bovine serum. HDAC small molecule substrate Boc-Lys(ac)-AMC was added to cell suspensions and incubated with cells for 90 minutes at 37° C. before reaction was stopped, and fluorescence was developed and read. As shown in FIG. 6, whole cell HDAC activity of human white blood cells was a function of cell numbers.

### Example 6

### Ex Vivo Inhibition of Whole Cell HDAC Activity (Class I or Class II) in Human White Blood Cells Using Boc-LyAc-AMC as Substrate

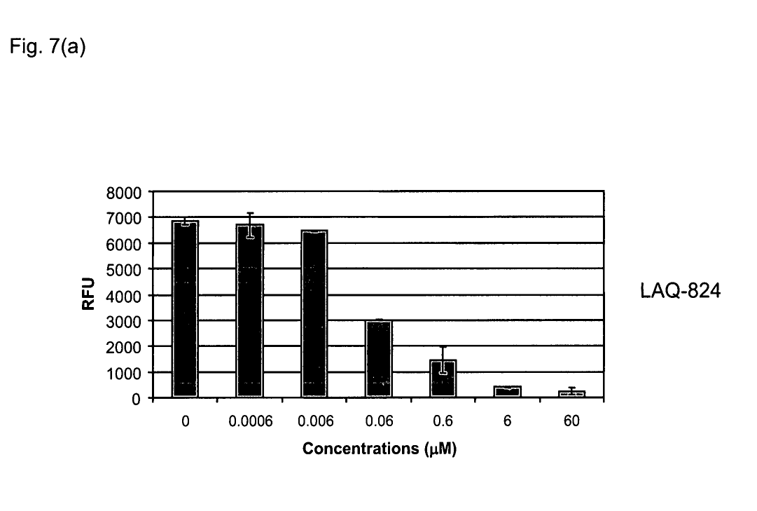

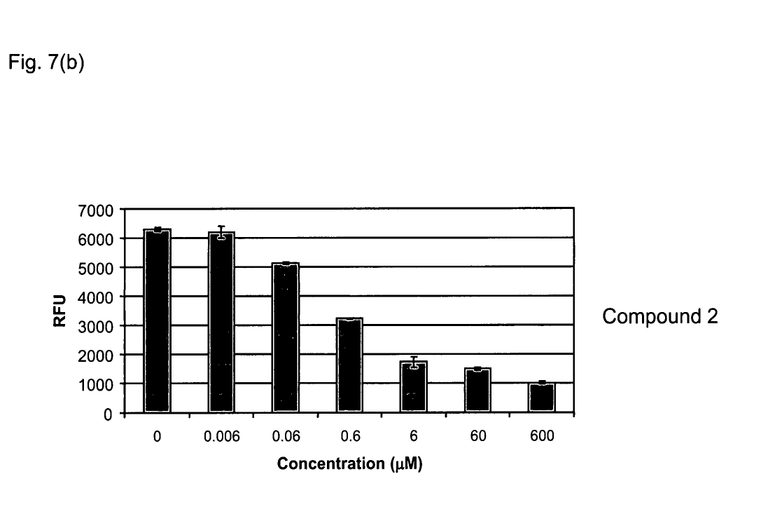

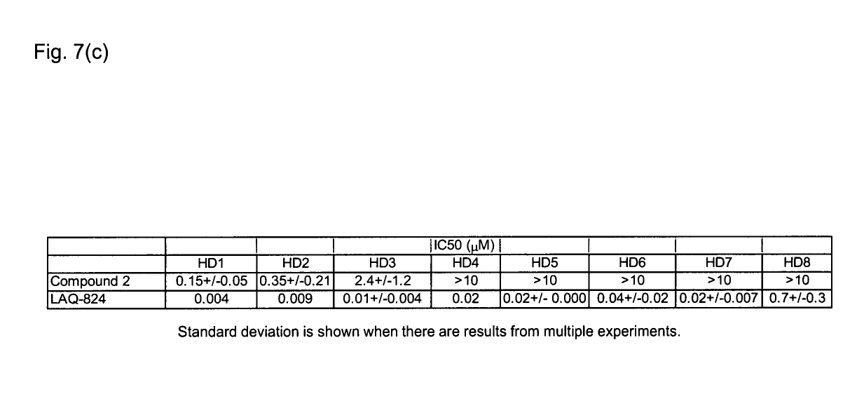

Human white blood cells (buffy coat) isolated from human donors were plated into 96-well dish in RPMI plus 10% fetal bovine serum. HDAC inhibitors with a range of dilutions were incubated with cells for 16 hours at 37 C with 5% CO₂. HDAC small molecule substrate Boc-Lys(ac)-AMC was added into cell suspensions and incubated with cells for 90 minutes before reaction was stopped, and fluorescence was developed and read. Both a pan-inhibitor (FIG. 7a; LAQ-824) and an isotype-specific inhibitor for HDACs1-3 (FIG. 7b; Compound 2) gave dose-dependent inhibition. FIG. 7c shows IC₅₀s (in μM) of these inhibitors against recombinant HDAC enzymes in vitro using the same small molecule substrate Boc-Lys(Ac)-AMC. 70% of total HDAC activity was inhibited by the isotype-specific inhibitor, indicating that HDACs 1-3 provide most of the activity in white blood cells from human.

### Example 7

### Time-Dependent Inhibition of HDAC Activity in White Blood Cells in Animals Treated with Compound 2 In Vivo

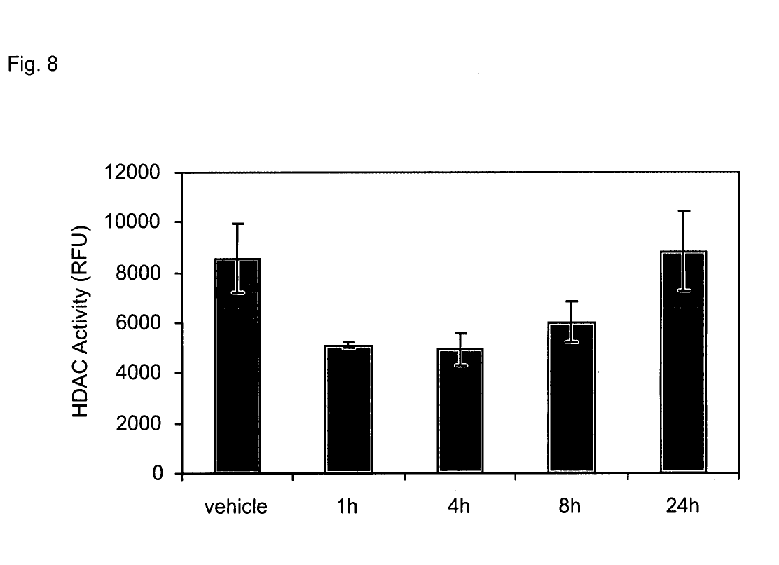

CD-1 mice (5 per group) were treated with either vehicle (PEG400:0.2N HCl in saline at 40:60 ratio) or Compound 2 at 90 mg/kg by oral administration for a single dose for indicated time period. Blood for each group of animals were arranged to harvest at the same point and were stored at 4 C overnight. White blood cells from individual animal were isolated. HDAC enzyme assay was performed using Boc-Lys (Ac)-AMC as described above. The results are shown in FIG. 8.

### Example 8

### Dose-Dependent Inhibition of Whole Cell HDAC Activity In Vivo

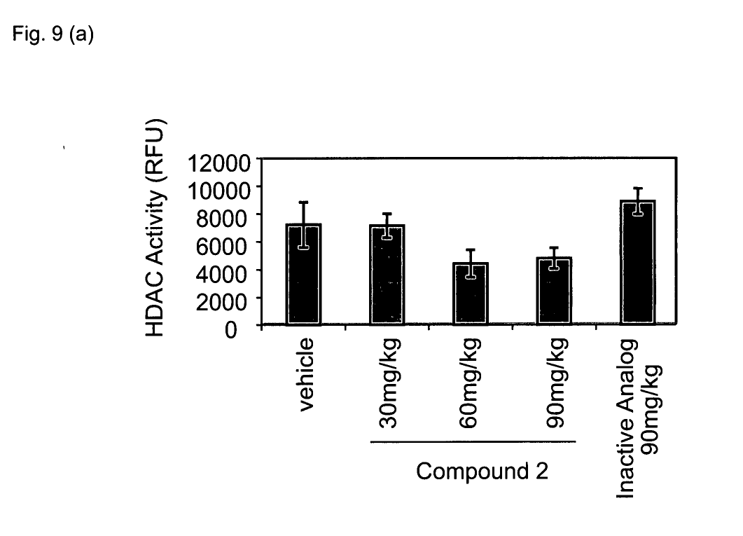

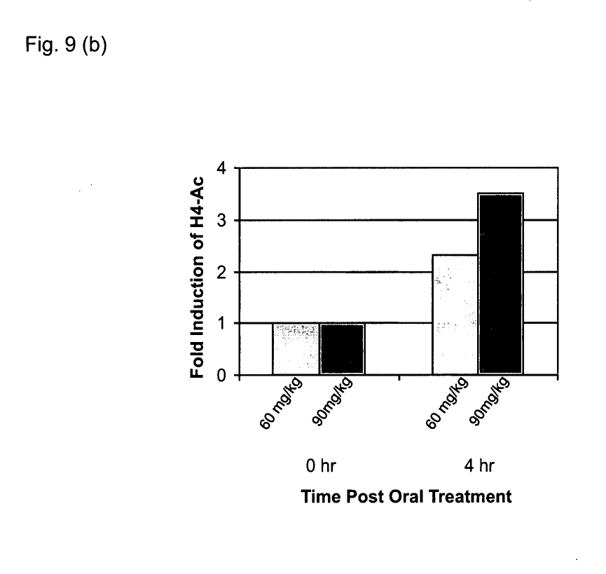

CD-1 mice (5 per group) were treated with either vehicle (PEG400:0.2N HCl in saline at 40:60 ratio) or Compound 2 or an inactive analog of Compound 2 (with similar molecular weight). Compounds were orally administered into mice at indicated single doses. Blood for each group of animals were harvested and stored at 4 C for overnight. White blood cells from individual animal were isolated. HDAC enzyme assay was performed using Boc-Lys (Ac)-AMC. Compound 2 but not its inactive analog inhibits HDAC activity in murine white blood cells in a dose-dependent manner (FIG. 9a).

### Example 9

### Dose and Time-Dependent Induction of Histone Acetylation In Vivo

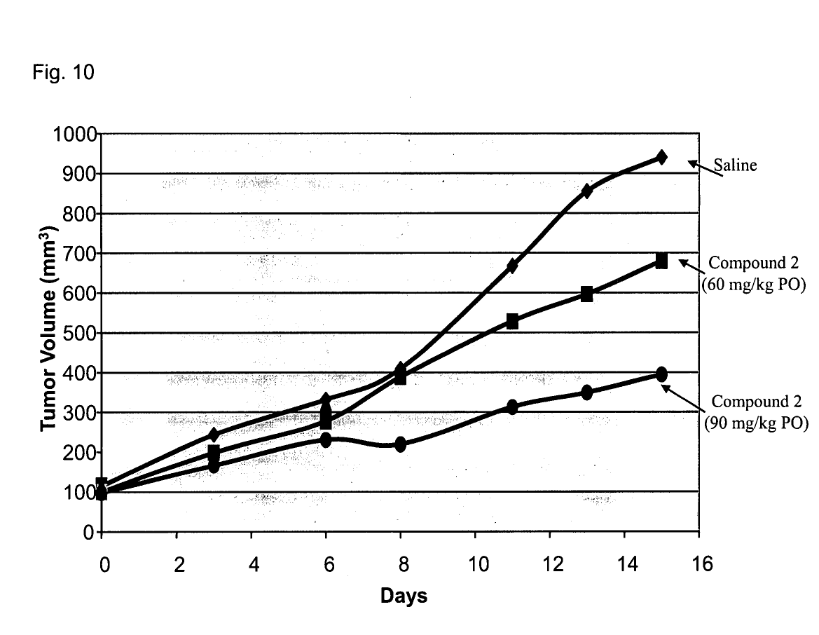

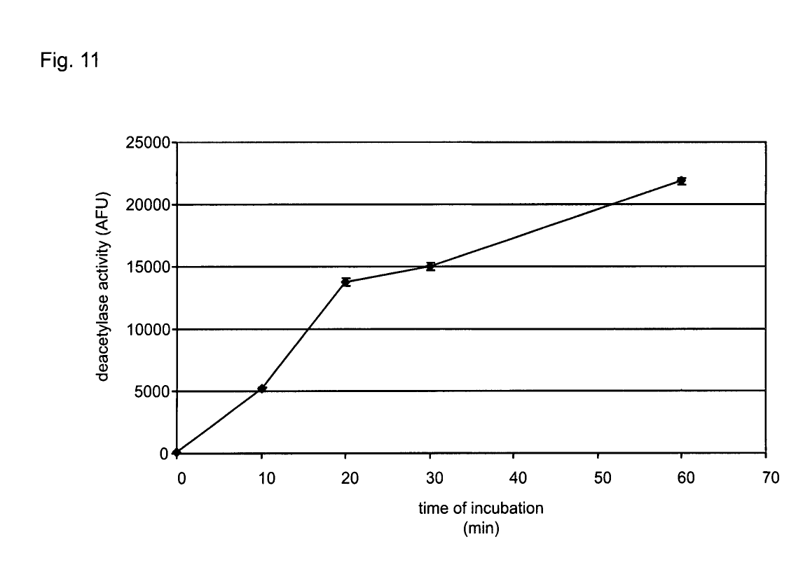

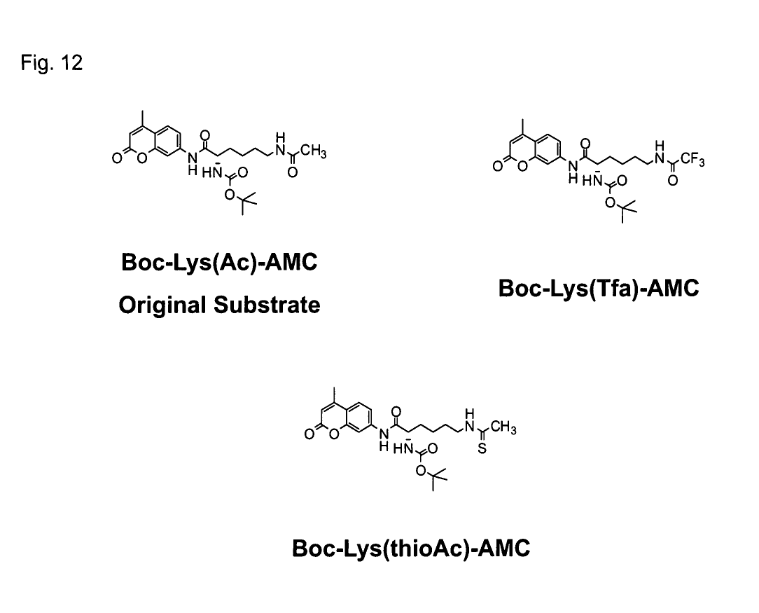

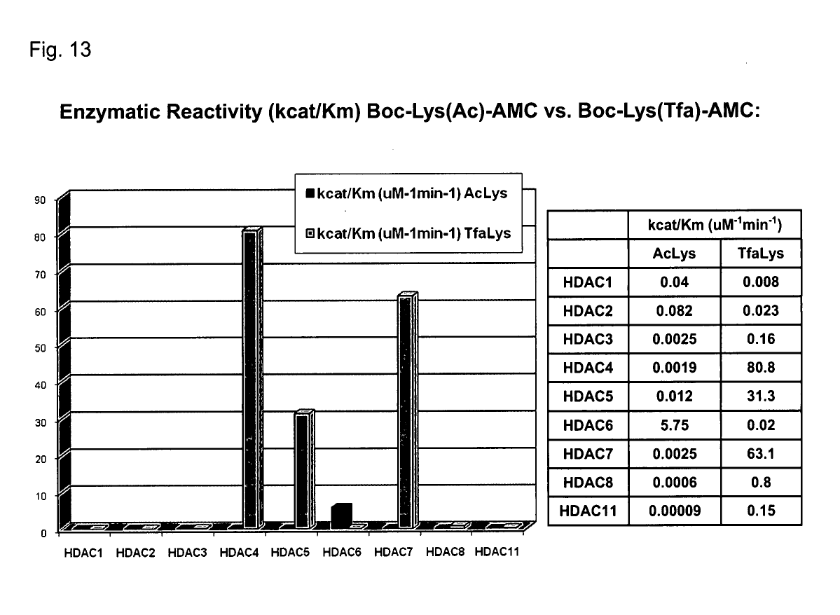

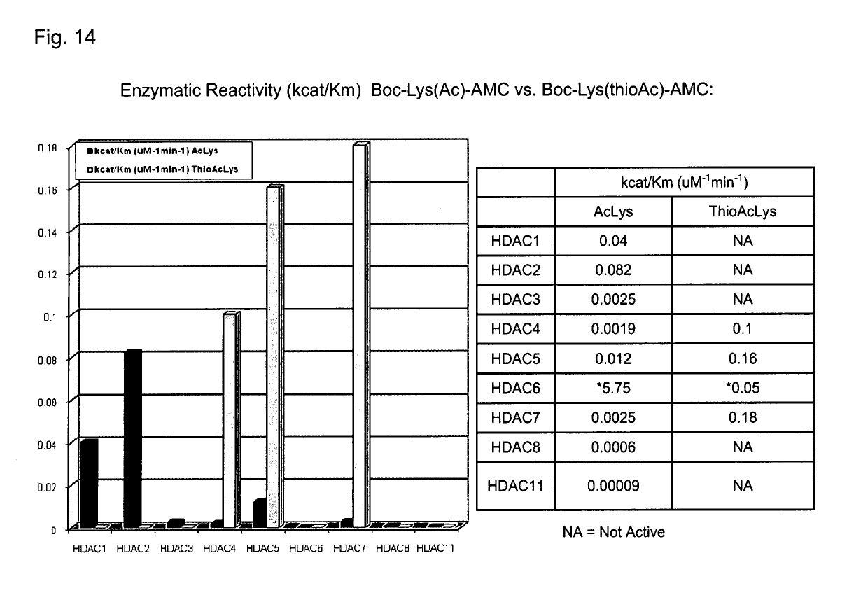

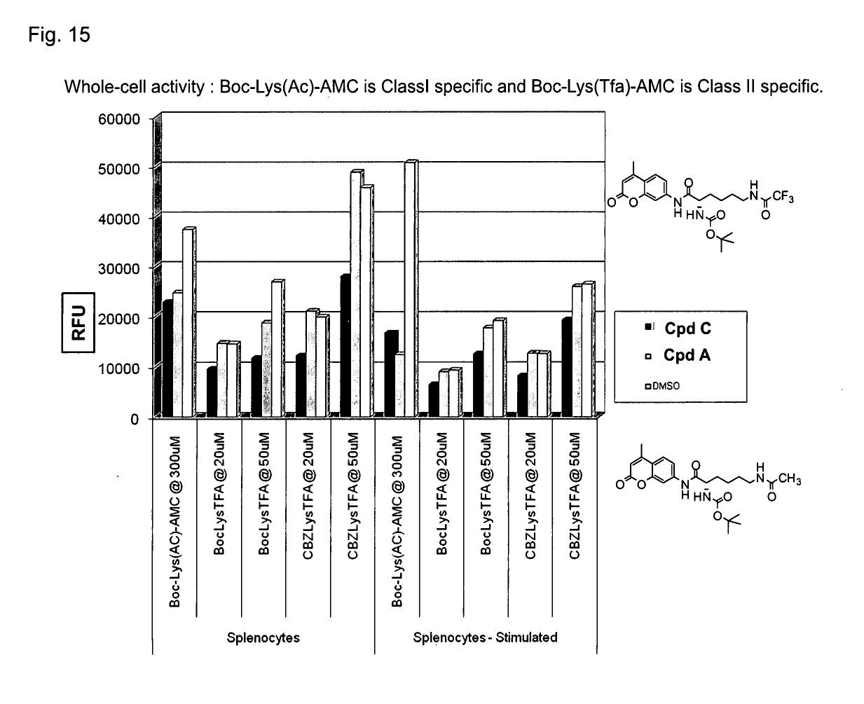

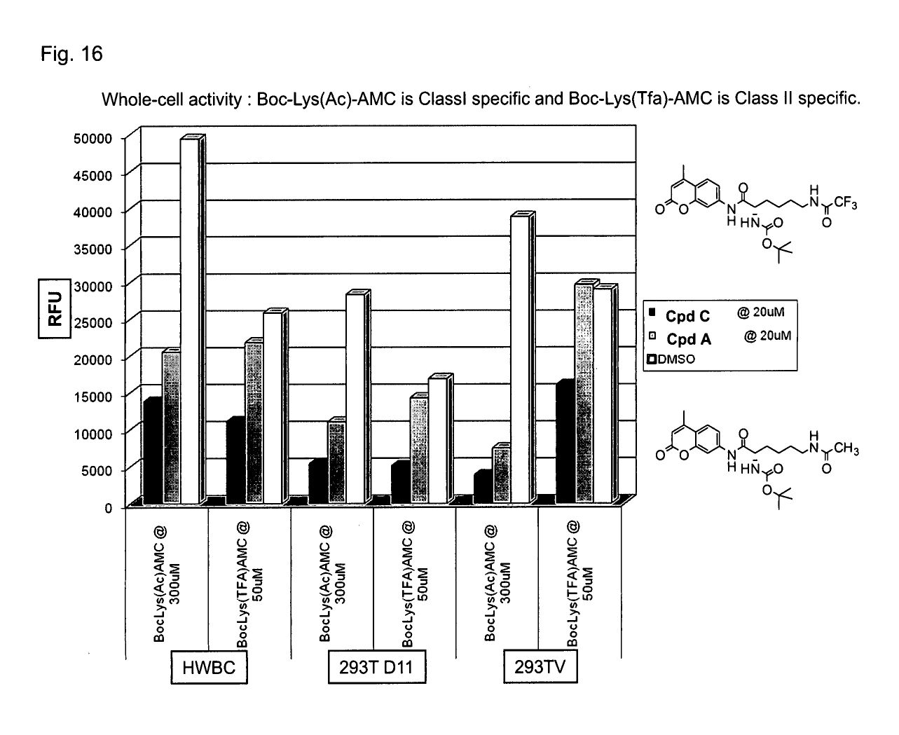

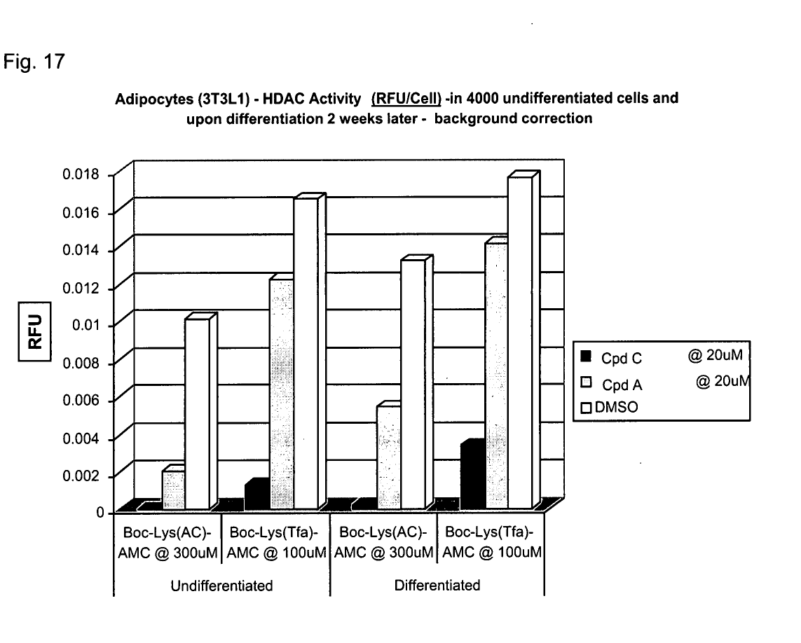

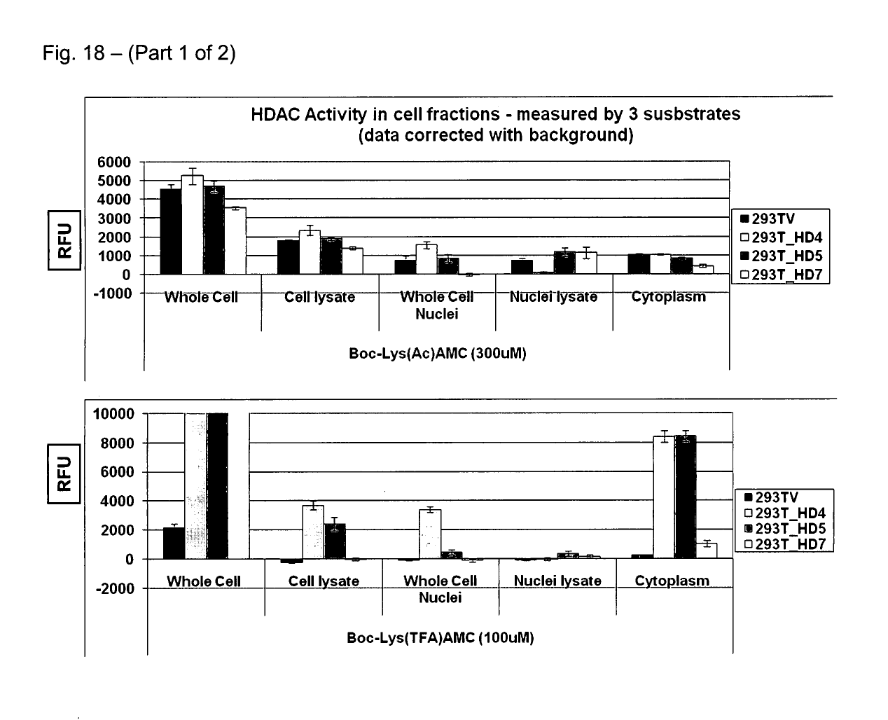

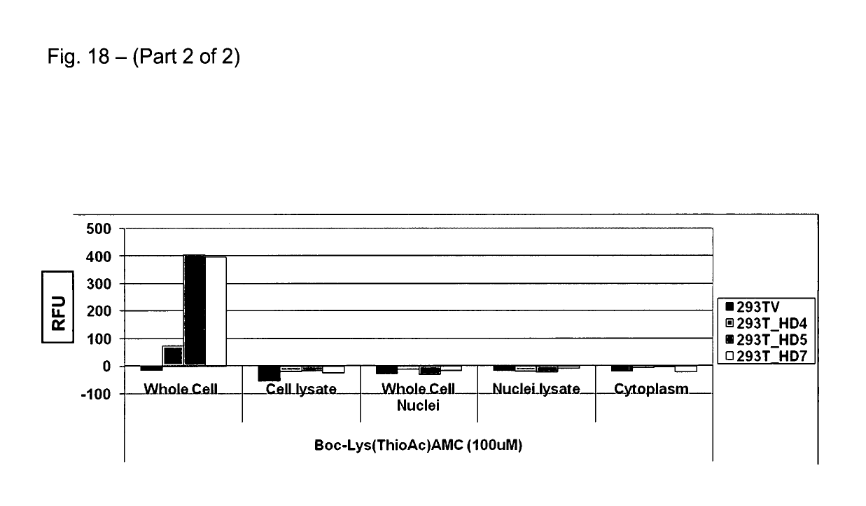

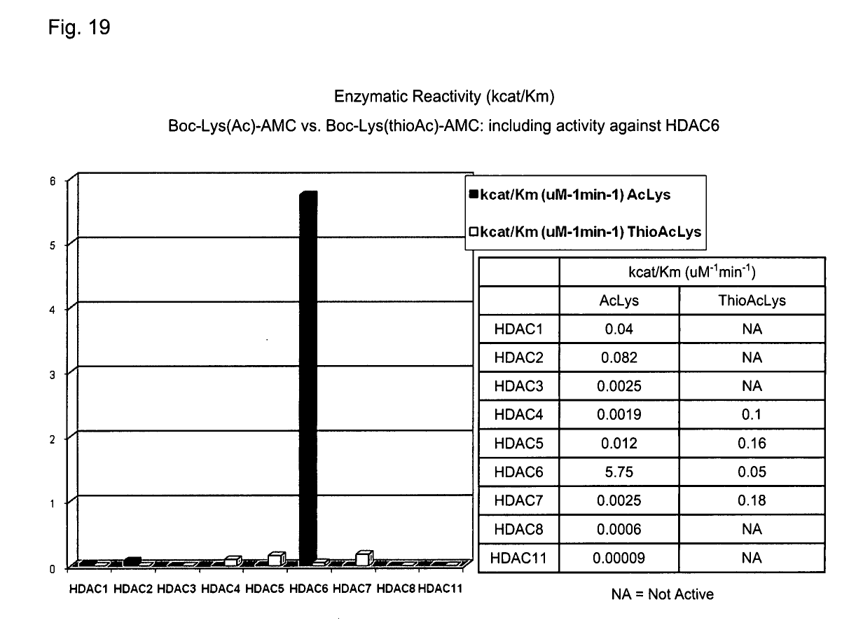

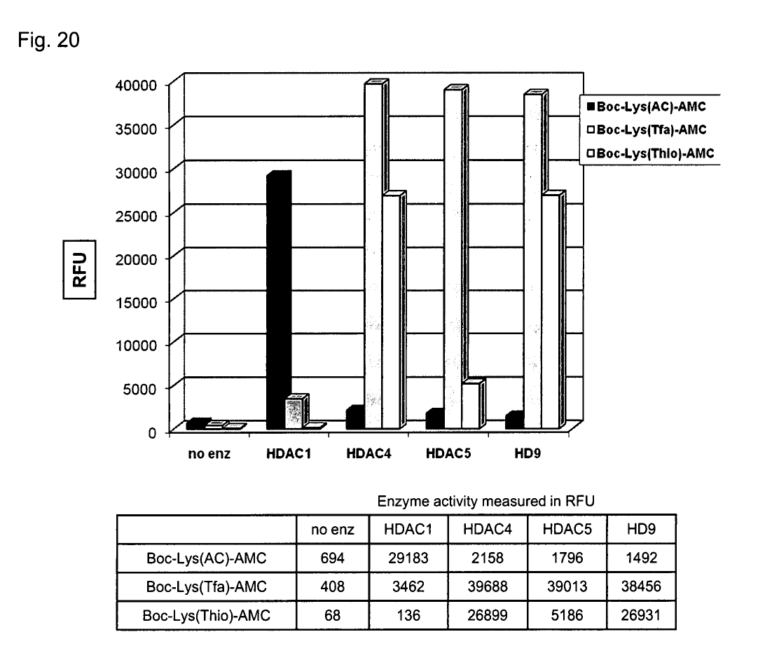

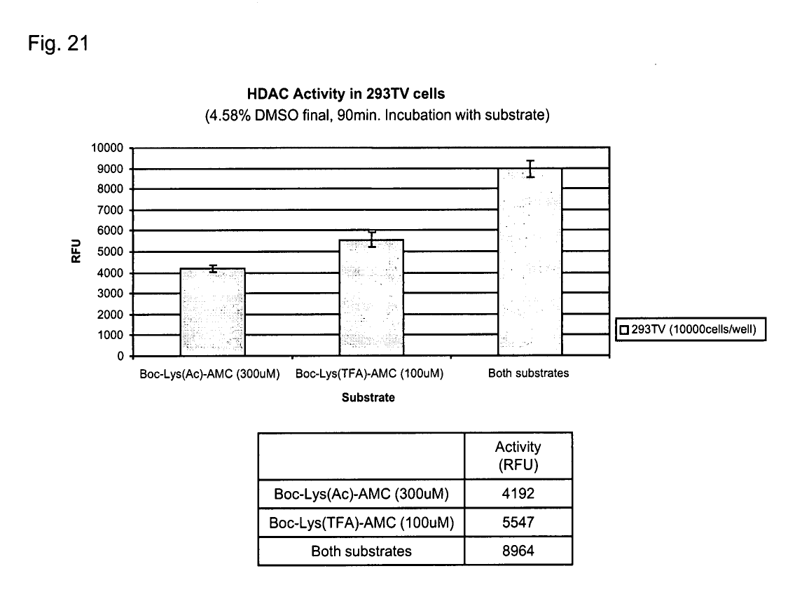

CD-1 nude mice (3 per group) were treated with either vehicle (PEG400:0.2N HCl in saline at 40:60 ratio) or Compound 2 (free base at 60 mg/kg or 90 mg/kg) by oral administration for 4 hours. Blood from each group were pooled and white blood cells were isolated. White blood cells (at least 2×10⁷) were lysed in ice-cold lysis buffer (10 mM Tris-HCl, pH 8.0, 1.5 mM MgCl₂, 5 mM KCl, 0.5% NP-40, 12 uM DTT, 5 mM Sodium butyrate and freshly prepared protease inhibitors). Cells were incubated on ice for 10 minutes and centrifuged at 2000 rpm for 15 minutes at 4° C. in a IEC Micromax centrifuge (Fisher Scientific Ltd., Nepean, Ontario). Pellets were washed one time with cold lysis buffer and cold concentrated H₂SO₄ acid (final 0.4 M) was added to cell pellets, and resuspended pellets were incubated on ice for at least one hour before they were centrifuged at 15000 rpm for 5 minutes at 4° C. Supernatant was transferred to a 15 ml polypropylene Falcon tube (Becton Dickinson Laboratories, Franklin Lakes, N.J.) and acetone (10× volumes of the supernatant) was added. Supernatant with acetone was incubated at −20° C. for overnight and histones were recovered by centrifugation at 2000 rpm for 5 minutes at 4° C. Acid-extracted histones were air dried and resuspended in water and protein concentration determined by using BioRad protein assay (Bio-Rad Laboratories (Canada) Ltd., Mississauga, Ontario). Histones from white blood cells were analyzed by SDS-PAGE followed by Western blot using anti-acetylated H4 histone or anti-histone H4 antibodies. Acetylation of H4 histone for each group were normalized against that of vehicle-treated group. Enzyme inhibition of HDAC activity by Compound 2 in blood correlated with its induction of histone acetylation (FIG. 9b). Interestingly, the dose where Compound 2 can inhibit 50% of enzyme activity in white blood cells (60 mg/kg) is approximately the dose which leads to significant anti-tumor activity in vivo (FIG. 10). Enzyme inhibition of HDAC by Compound 2 correlated with its antitumor activity in mice (see below).

### Example 10

### Dose-Dependent Antitumor Activity of Compound 2 in A431 Human Epidermoid Carcinoma Xenograft Model in Nude Mice

CD-1 Nude Mice bearing human A431 tumors (8 per group) were treated with either saline alone or various doses of Compound 2 in PEG400:0.2N HCl in saline at 40:60 ratio daily by oral administration. Briefly, A431 cells (2 million) were injected subcutaneously in the animal flank and allowed to form solid tumors. Tumor fragments were passaged in nude mice for a minimum of three times before their use. Tumor fragments (about 30 mg) were implanted subcutaneously through a small surgical incision under general anesthesia to CD1 female nude mice (6-8 weeks old, from Charles River Laboratories, Wilmington, Mass.). Recipient animals were treated with saline or HDAC inhibitors by oral administrations when the tumor sizes reached about 100 mm³. Tumor volumes and gross body weight of animals were monitored twice weekly for up to 2 weeks. Each experimental group contained at least 8 animals. Student's Tests were used to analyze the statistical significance between numbers in data sets. Tumor volumes were monitored for 2 weeks. The results are shown in FIG. 10.

### Example 11

### Assessment of Deacetylase Activity Using Bodily Fluids

CD-1 Mouse blood was collected in heparin tubes and cells were counted by Coulter counter (Beckman Coulter, Ville St. Laurent, Quebec). The amount of whole blood containing 1.6×10E6 white blood cells was aliquoted and the volume was brought up to 200 ul with RPMI (+10% FBS). Boc-Ac-Lys-AMC was added to a final concentration of 300 uM. After various amounts of time, the mix was spun (400×g for 5 min), and 50 ul of the supernatant (serum) was transferred to a 96-well plate. The amount of deacetylated product Boc-Lys-AMC present in the supernatant was detected by adding an equal volume of the developer mix and reading after 15 minutes incubation. The results are shown in FIG. 11. This finding is consistent with our observation in Example 1, where not only the substrate Boc-Lys(Ac)-AMC is permeable to go inside cells, but also the deacetylated product Boc-Lys-AMC is permeable to come out from cells. Thus total HDAC activity in primary cells could be easily monitored in bodily fluid where animals were contacted with HDAC substrates ex vivo.

### Example 12

### Assessment of Protein Deacetylase Activity In Vivo Using Bodily Fluids

CD-1 mice (6 per group) or rats (6 per group) are treated with a cell permeable pan-substrate at 1 to 100 mg/kg by a single i.v. administration. Three of the mice (or rats) are then treated with a pan-inhibitor of a protein deacetylase family. At times thereafter, blood is taken, plasma separated and analyzed for the quantity of the detectable reporter molecule. The quantity of reporter molecule in the plasma from inhibitor-treated mice is compared with the quantity in the plasma of the untreated mice.

### Example 13

### Class II HDAC Enzyme Assay Using Boc-Lys(TFA)-AMC

A 30 mM stock of Boc-Lys(TFA)-AMC substrate was prepared in DMSO. 2 μl test compound in DMSO was diluted in 50 μl buffer (25 mM HEPES, pH 8.0, 137 mM NaCl, 2.7 mM KCl, 1 mM MgCl₂, 0.1% BSA) and pre-incubated with Class II HDAC enzyme (30 μl of a final enzyme concentration of 0.1-0.2 nM) for 10 minutes at room temperature. Reaction was started by adding 18 μl Boc-Lys(TFA)-AMC substrate and incubating at 37° C. for 20-30 minutes. The reaction was stopped by adding 50 μl trypsin (1 mg/ml) and a known HDAC inhibitor. The plate was then incubated in the dark for 20 minutes at room temperature and read with Ex=360 nm, Em=470 nm, cutoff filter at 435 nm.

### Example 14

### Class II HDAC Enzyme Assay Using Boc-Lys(thioAc)-AMC

A 30 mM stock of Boc-Lys(thioAc)-AMC substrate was prepared in DMSO. 2 μl test compound in DMSO was diluted in 15 μl buffer (25 mM HEPES, pH 8.0, 137 mM NaCl, 2.7 mM KCl, 1 mM MgCl₂, 0.1% BSA) and pre-incubated with Class II HDAC enzyme (15 μl of a final enzyme concentration of 50-500 nM) for 10 minutes at room temperature. Reaction was started by adding 18 μl Boc-Lys(thioAc)-AMC substrate and incubating at 37° C. for 30-60 minutes. The reaction was stopped by adding 50 μl trypsin (1 mg/ml) and a known HDAC inhibitor. The plate was then incubated in the dark for 20 minutes at room temperature and read with Ex=360 nm, Em=470 nm, cutoff filter at 435 nm.

### Example 15

### Class II HDAC Whole Cell Assay Using Boc-Lys(ThioAc)-AMC

293TV, 293T-HD4, 293T-HD5 and 293T-HD7 cells were plated at respective concentrations of 10,000 cells/well, 2,000 cells/well, 1,000 cells/well and 500 cells/well in 96-well culture plates in a final volume of 80 μl media with a dispenser system (MultiDrop). Plates were incubated overnight at 37° C. under a 5% CO₂ atmosphere. Test compounds were dissolved in DMSO at 30 mM and serial dilutions (0.005-50 μM) were made with a liquid handling system (TECAN) in 100% DMSO. 5 μl of each serial dilution was diluted in 45 μl of a 25% DMSO/DMEM solution with a robot (BioMek FX, Beckman), final concentration of DMSO of 32.5%. Then, 5 μl of each intermediate dilution was transferred to cell plates with the robot (final DMSO concentration of 1.9%). Plates were incubated for 2-17 hours at 37° C. under a 5% CO₂ atmosphere. After this incubation, 5 μl of 1.8 mM Boc-Lys(TFA)-AMC (100 μM final concentration) substrate was added by the robot to each well and plates were incubated for 90 minutes at 37° C., 5% CO₂. Reaction was stopped by addition of 50 μl trypsin (1 mg/ml), 1% NP40, 2 μl quench (known inhibitor, e.g., Cpd C) solution mix. Plates were then incubated in the dark at room temperature for 20 minutes. Plates were then read using a Gemini fluorescence reader at Ex 360 nm, Em 470 nm, with a 435 nm cutoff filter.

### EQUIVALENTS

Those skilled in the art will recognize, or be able to ascertain, using no more than routine experimentation, many equivalents to the specific embodiments of the invention described herein. Such equivalents are intended to be encompassed by the following claims.

1. A method for assessing Class II histone deacetylase activity or one or more member thereof in whole cells ex vivo, or in extracts of such cells or in extracts of subcellular compartments from such cells comprising providing whole cells from a mammal, contacting the whole cells with a cell-permeable Class II histone deacetylase-specific substrate, wherein deacetylation of the substrate by Class II histone deacetylases or the one or more member thereof generates a detectable reporter molecule, and measuring the quantity of the detectable reporter molecule either in the whole cells, or in extracts from such cells or extracts from subcellular compartments of such cells.

2. The method of claim 1, wherein the quantity of the detectable reporter molecule is measured against a control standard for the Class II HDAC family or the one or more member thereof.

3. A method for assessing isotype-specific activity of one or more member of the Class II histone deacetylase family from whole cells ex vivo, or in extracts from such cells or extracts from subcellular compartments of such cells, wherein one or more isotype of the Class II HDAC family provides a majority of the total deacetylase activity, the method comprising providing whole cells from a mammal, contacting the whole cells with a cell-permeable Class II HDAC-specific substrate or a cell permeable isotype-specific substrate for the one or more member of the Class II HDAC family, wherein deacetylation of the substrate by the one or more Class II HDAC generates a detectable reporter molecule, contacting a first aliquot of the cells with an isotype-specific inhibitor of the one or more Class II HDAC that provides a majority of the total HDAC activity, not contacting a second aliquot of the cells with the isotype-specific inhibitor of the one or more Class II HDAC that provides a majority of the total HDAC activity, measuring the quantity of the detectable reporter molecule for the first and second aliquots and comparing the quantity of HDAC activity for each aliquot.

4. The method of claim 3, wherein the quantity of the detectable reporter molecule is measured against a control standard for the Class II HDAC family or the one or more member thereof. A method for assessing the activity of a specific isotype of one or more member of the Class II HDAC family in cells ex vivo, in extracts of such cells, or in extracts of sub-cellular compartments of such cells, the method comprising providing whole cells from a mammal, contacting the whole cells with a cell-permeable isotype-specific substrate for the one or more member of the Class II HDAC family, wherein deacetylation of the substrate by the HDAC generates a detectable reporter molecule, and measuring the quantity of the detectable reporter molecule.

5. The method of claim 9, wherein the quantity of the detectable reporter molecule is measured against a control standard for the one or more members of the Class II HDAC family.

6. A method for assessing the activity of a candidate Class II HDAC-specific inhibitor or an inhibitor of one or more member thereof in whole cells ex vivo, in extracts of such cells, or in extracts of sub-cellular compartments of such cells, the method comprising providing whole cells from a mammal, contacting the whole cells with a cell-permeable candidate Class II HDAC-specific substrate, wherein deacetylation of the substrate by the Class II HDAC family or one or more members thereof generates a detectable reporter molecule, contacting a first aliquot of the cells with a candidate Class II HDAC-specific inhibitor, not contacting a second aliquot of the cells with the candidate Class II HDAC-specific inhibitor, measuring the quantity of the detectable reporter molecule for the first and second aliquots and comparing the quantity of protein deacetylase activity for each aliquot.

7. The method of claim 6, wherein the quantity of the detectable reporter molecule is measured against a control standard for the Class II HDAC family or one or more members thereof.

8. A method for assessing isotype-specific activity of a candidate inhibitor of a member of the Class II HDAC family from whole cells ex vivo, in extracts of such cells, or in extracts of sub-cellular compartments of such cells, wherein one or more isotype of the HDAC Class II family provides a majority of the total deacetylase activity, the method comprising providing whole cells from a mammal, contacting the whole cells with a cell-permeable Class II HDAC-specific substrate or a cell permeable isotype-specific substrate for one or more member of the Class II HDAC family, wherein deacetylation of the substrate by the protein deacetylase generates a detectable reporter molecule, contacting a first aliquot of the cells with the candidate isotype-specific inhibitor of the HDAC isotype that provides a majority of the total deacetylase activity, not contacting a second aliquot of the cells with the candidate isotype-specific inhibitor of the HDAC isotype that provides a majority of the total deacetylase activity, measuring the quantity of the detectable reporter molecule for the first and second aliquots, and comparing the quantity of the detectable reporter molecule for each aliquot.

9. The method of claim 8 wherein the quantity of the detectable reporter molecule is measured against a control standard for the HDAC isotype that provides a majority of the total deacetylase activity.

10. A method for assessing the efficacy of a Class II HDAC-specific inhibitor or an inhibitor of one or more member thereof in vivo, the method comprising providing whole cells from a mammal, contacting the cells with a Class II HDAC-specific substrate or an isotype specific substrate, wherein deacetylation of the substrate by the Class II HDAC family or one or more members thereof generates a detectable reporter molecule, determining the quantity of the reporter molecule, administering the Class II HDAC-specific inhibitor to the mammal, taking whole cells from the mammal, contacting the whole cells with the Class II HDAC-specific substrate, determining the quantity of the reporter molecule, and comparing the quantity of the reporter molecule after administration of the Class II HDAC-specific inhibitor with the quantity of the reporter molecule before administration of the Class II HDAC-specific inhibitor, wherein a decrease in the quantity of the reporter molecule after administration of the Class II HDAC-specific inhibitor is taken as a measure of efficacy.

11. The method according to claim 10, wherein, the quantity of reporter molecule from the whole cells is standardized against a known activity of the Class II HDAC family or the one or more members thereof.

12. A method for assessing the efficacy and specificity of an isotype-specific inhibitor of one or more member of the Class II HDAC family in vivo, the method comprising providing whole cells from a mammal, contacting the whole cells with an isotype-specific substrate for the one or more member of the Class II HDAC family, wherein deacetylation of the substrate by the HDAC generates a detectable reporter molecule, determining the quantity of the reporter molecule, administering to the mammal the isotype-specific inhibitor, taking whole cells from the mammal, contacting the whole cells with the isotype-specific substrate, determining the quantity of the reporter, and comparing the quantity of the reporter molecule after administration of the isotype-specific inhibitor with the quantity of the reporter molecule before administration of the isotype-specific inhibitor, wherein a decrease in the quantity of the reporter molecule after administration of the isotype-specific inhibitor is taken as a measure of efficacy.

13. The method according to claim 12, wherein the quantity of the reporter molecule from the whole cells is standardized against a known activity of the one or more member of the Class II HDAC family.

14. A method for assessing the efficacy of a Class II HDAC-specific inhibitor or an inhibitor of one or more member thereof in vivo by measuring the quantity of a detectable reporter molecule in bodily fluids from a mammal, the method comprising administering to the mammal a cell-permeable Class II HDAC-specific substrate or an isotype-specific substrate, wherein deacetylation of the Class II HDAC-specific substrate or isotype-specific substrate generates a detectable reporter molecule, obtaining bodily fluids from the mammal, determining the quantity of the detectable reporter molecule in the bodily fluids, administering to the mammal a Class II HDAC-specific inhibitor, administering to the mammal the Class II HDAC-specific substrate, obtaining bodily fluids from the mammal, determining the quantity of the detectable reporter molecule in the bodily fluids, and comparing the quantity of detectable reporter molecule in bodily fluids obtained prior to administration of the Class II HDAC-specific inhibitor with the quantity of the detectable reporter molecule in bodily fluids after administration of the Class II HDAC-specific inhibitor, wherein a decrease in the quantity of the reporter molecule after administration of the Class II HDAC-specific inhibitor is taken as a measure of efficacy.

15. A method for assessing the efficacy of an isotype-specific inhibitor of one or more member of the Class II HDAC family in a mammal in vivo by measuring the quantity of a detectable reporter molecule in bodily fluids, the method comprising administering to the mammal a cell-permeable isotype-specific substrate for the one or more member of the Class II HDAC family, wherein deacetylation of the isotype-specific substrate generates the detectable reporter molecule, obtaining bodily fluids from the mammal, determining the quantity of the detectable reporter molecule in the bodily fluids, administering to the mammal an isotype-specific inhibitor of one or more member of the Class II HDAC family, administering to the mammal the isotype-specific substrate, obtaining bodily fluids from the mammal, determining the quantity of the detectable reporter molecule in the bodily fluids, and comparing the quantity of detectable reporter molecule in bodily fluids obtained prior to administration of the isotype-specific inhibitor with the quantity of the detectable reporter molecule in bodily fluids after administration of the isotype-specific inhibitor, wherein a decrease in the quantity of the reporter molecule after administration of the isotype-specific inhibitor is taken as a measure of efficacy.

16. A method for assessing the efficacy of a Class II HDAC-specific activator or an activator of one or more member thereof in vivo, the method comprising providing whole cells from a mammal, contacting the cells with a Class II HDAC-specific substrate or an isotype specific substrate, wherein deacetylation of the substrate by the Class II HDAC family or one or more members thereof generates a detectable reporter molecule, determining the quantity of the reporter molecule in the whole cells, administering to the mammal the Class II HDAC-specific activator or activator of one or more member thereof, taking whole cells from the mammal, contacting the whole cells with the Class II HDAC-specific substrate, determining the quantity of the reporter molecule, and comparing quantity of the reporter molecule after administration of the Class II HDAC-specific activator with the quantity of the reporter molecule before administration before administration of the Class II HDAC-specific activator, wherein an increase in the quantity of the reporter molecule after administration of the Class II HDAC-specific activator is taken as a measure of efficacy.

17. The method according to claim 16, wherein the quantity of the reporter molecule is standardized against a known activity of the Class II HDAC family or the one or more members thereof.

18. A method for assessing the efficacy and specificity of an isotype-specific activator of one or more member of the Class II HDAC family in vivo, the method comprising providing whole cells from a mammal, contacting the cells with an isotype-specific substrate for the one or more member of the Class II HDAC family, wherein deacetylation of the substrate by the HDAC generates a detectable reporter molecule, determining the quantity of the reporter molecule, administering to the mammal the isotype-specific activator, taking whole cells from the mammal, contacting the whole cells with the isotype-specific substrate, determining the quantity of the reporter molecule in the whole cells, comparing the quantity of the reporter molecule after administration of the isotype-specific activator with the quantity of the reporter molecule before administration of the isotype-specific activator, wherein an increase in the quantity of the reporter molecule after administration of the isotype-specific activator is taken as a measure of efficacy.

19. The method according to claim 18, wherein the quantity of the reporter molecule is standardized against a known activity of the member of the Class II HDAC family.

20. A method for assessing the efficacy of a Class II HDAC-specific activator or an activator of one or more members thereof in vivo by measuring the quantity of a detectable reporter molecule in bodily fluids from a mammal, the method comprising administering to the mammal a cell-permeable Class II HDAC-specific substrate or substrate for one or more members thereof, wherein deacetylation of the Class II HDAC-specific substrate or isotype-specific substrate generates the detectable reporter molecule, obtaining bodily fluids from the mammal, determining the quantity of the detectable reporter molecule in the bodily fluids, administering to the mammal the Class II HDAC-specific activator, administering to the mammal the Class II HDAC-specific substrate or isotype specific substrate, obtaining bodily fluids from the mammal, determining the quantity of the detectable reporter molecule in the bodily fluids, and comparing the quantity of detectable reporter molecule in bodily fluids obtained prior to administration of the Class II HDAC-specific activator with the quantity of the detectable reporter molecule in bodily fluids after administration of the Class II HDAC-specific activator, wherein an increase in the quantity of the reporter molecule after administration of the Class II HDAC-specific activator is taken as a measure of efficacy.

21. A method for assessing the efficacy of an isotype-specific activator of one or more member of the Class II HDAC family in mammals in vivo by measuring the quantity of a detectable reporter molecule in bodily fluids of a mammal, the method comprising administering to the mammal a cell-permeable isotype-specific substrate for a Class II HDAC, wherein deacetylation of the isotype-specific substrate generates the detectable reporter molecule, obtaining bodily fluids from the mammal, determining the quantity of the detectable reporter molecule in the bodily fluids, administering to the mammal an isotype-specific activator of one or more member of the Class II HDAC family, administering to the mammal isotype-specific substrate, obtaining bodily fluids from the mammal, determining the quantity of the detectable reporter molecule in the bodily fluids, and comparing the quantity of detectable reporter molecule in bodily fluids obtained prior to administration of the isotype-specific activator with the quantity of the detectable reporter molecule in bodily fluids after administration of the isotype-specific activator, wherein an increase in the quantity of the reporter molecule after administration of the isotype-specific activator is taken as a measure of efficacy.

22. A method for assessing the activity of a candidate Class II HDAC-specific activator or an activator of one or more members thereof in whole cells ex vivo, the method comprising providing whole cells from a mammal, contacting the whole cells with a cell-permeable Class II HDAC-specific substrate or an isotype-specific substrate, wherein deacetylation of the substrate by the Class II HDAC family or one or more members thereof generates a detectable reporter molecule, contacting a first aliquot of the cells with a candidate Class II HDAC-specific activator, not contacting a second aliquot of the cells with the candidate Class II HDAC-specific activator, determining the quantity of the detectable reporter molecule in the first and second aliquots and comparing the quantity of Class II HDAC-specific activity for each aliquot.

23. The method according to claim 22, wherein the quantity of the detectable reporter molecule is measured against a control standard for the Class II HDAC family or the one or more members thereof.

24. A method for assessing the activity of a candidate isotype-specific activator of a member of the Class II HDAC family ex vivo, the method comprising providing whole cells from a mammal, contacting the whole cells with a cell-permeable isotype-specific substrate, wherein deacetylation of the substrate by the protein deacetylase family or one or more members thereof generates a detectable reporter molecule, contacting a first aliquot of the cells with a candidate isotype-specific activator of one or more member of the Class II HDAC family, not contacting a second aliquot of the cells with the candidate isotype-specific activator of one or more member of the Class II HDAC family, determining the quantity of the detectable reporter molecule for the first and second aliquots, and comparing the quantity of isotype-specific HDAC activity for each aliquot.

25. A method for assessing Class II histone deacetylase activity, or activity of one or more member thereof in a mammal in vivo by measuring the quantity of a detectable reporter molecule in bodily fluids, the method comprising administering to the mammal a cell-permeable Class II histone deacetylase-specific substrate, wherein deacetylation of the substrate by Class II histone deacetylases, or one or more member thereof generates a detectable reporter molecule, obtaining bodily fluids from the mammal, and determining the quantity of the detectable reporter molecule in the bodily fluids.

26. The method according to claim 25, further comprising measuring the quantity of the detectable reporter molecule against a control standard for the Class II histone deacetylase family or the one or more member thereof.

27. A method for assessing isotype-specific activity of one or more member of the Class II histone deacetylase family in a mammal in vivo by measuring the quantity of a detectable reporter molecule in bodily fluids, the method comprising administering to the mammal a cell-permeable Class II HDAC-specific substrate or a cell permeable isotype-specific substrate for the one or more member of the Class II HDAC family, wherein deacetylation of the substrate by the one or more member of the Class II HDAC family generates a detectable reporter molecule, obtaining a first sample of bodily fluid, administering to the mammal an isotype-specific inhibitor of the one or more Class II HDAC, obtaining a second sample of bodily fluid, measuring the quantity of the detectable reporter molecule for the first and second samples, and comparing the quantity of HDAC activity for each sample.

28. The method according to claim 27, further comprising measuring the quantity of the detectable reporter molecule against a control standard for the Class II histone deacetylase family or the one or more member thereof.

29. A compound of formula (I), provided that the compound is not Boc-Lys(Ac)-AMC or Boc-Lys(Tfa)-AMC: 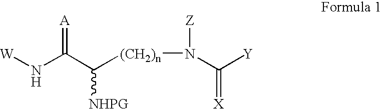
wherein
X is selected from the group consisting of O, S, NH and N(alkyl);
Y is selected from the group consisting of OH, alkoxy, alkyl, alkenyl and alkynyl, each of which alkoxy, alkyl, alkenyl and alkynyl, is optionally substituted with 1 to 7 substituents independently selected from the group consisting of halo, cyano, alkoxy, alkylamino and alkylthio;
Z is selected from the group consisting of H, alkyl, alkenyl and alkynyl;
n is an integer ranging from 0 to 12;
PG is a protecting group (preferably selected from the group consisting of MECO, CF₃CO—, Boc and CBZ), an amino acide or a peptide;
A is selected from the group consisting of O, S, NH and N(alkyl); and
W is a carboxycylic, heterocyclic, saturated or unsaturated, aromatic or heteroaromatic mono-, bi-, tri- or tetracyclic ring system.

30. The use of a compound according to claim 29 as a substrate for Class II histone deacetylases.

31. A complex of a compound according to claim 29 bound to a Class II histone deacetylase.

32. The method according to claim 24, wherein the quantity of the detectable reporter molecule is measured against a control standard for the member of the Class II HDAC family.
